(19) United States
(12) Patent Application Publication
(10) Pub. No.: US 2026/0199384 A1
(43) Pub. Date: Jul. 16, 2026

(54) METHOD AND COMPOSITIONS FOR TREATING SKIN DISORDERS

(71) Applicant: PHOENIX ANIMAL WELLNESS, INC., SAN ANTONIO, TX (US)

(72) Inventors: Theresa W. FOSSUM, Carefree, AZ (US); Jose R. MATOS, Plano, TX (US); Robert A. NEWMAN, Cape Elizabeth, ME (US)

(73) Assignee: PHOENIX ANIMAL WELLNESS, INC., SAN ANTONIO, TX (US)

(21) Appl. No.: 19/418,149

(22) Filed: Dec. 12, 2025

Related U.S. Application Data
(63) Continuation of application No. PCT/US2024/033954, filed on Jun. 14, 2024.
(60) Provisional application No. 63/507,804, filed on Jun. 13, 2023.

(51) Int. Cl. A61K 31/7048; A61K 31/519; A61K 36/24; A61K 45/06; A61P 17/04

(52) U.S. Cl. CPC A61K 31/7048; A61K 31/519; A61K 36/24; A61K 45/06; A61P 17/04; A61K 2236/331; A61K 2236/37

(57) ABSTRACT

A method of treating or preventing pruritus or itch-related skin disorder in a subject is provided. Related cardiac glycoside-containing compositions are also provided.

### CROSS-REFERENCE TO EARLIER FILED APPLICATIONS

The present application claims the benefit of priority and is a continuation-in-part of international application No. PCT/US2024/33954 filed Jun. 14, 2024, which claims the benefit of priority of application No. 63/507,804 filed Jun. 13, 2023, the entire disclosures of which are hereby incorporated by reference.

### FIELD OF THE INVENTION

The present invention concerns a method of treating skin disorders by administration of a cardiac glycoside, in particular, oleandrin and/or digoxin, to a subject in need thereof. In particular, pruritus and itch-related skin disorders are treated and/or prevented by administration of oleandrin-containing composition, optionally in combination with one or more other components. The subject may be an animal or human.

### BACKGROUND OF THE INVENTION

Skin disorders that occur in domestic animals or those of commercial importance are common, particularly among dogs, cats, horses, and cows. Exemplary skin disorders include pruritus, urticaria, xerosis (dry skin), eczema (atopic dermatitis), psoriasis, hives, welts, nettle rash. Pruritus (a.k.a. pruritis) may be associated with allergic dermatitis (caused by allergy to allergens, e.g. fleas, dust mites, grass, plant pollens, chemicals, etc.), contact dermatitis, or food hypersensitivity dermatitis. Many of these disorders have no suitable treatments.

Particular cytokines of the immune system are associated with skin disorders. Interleukin-17 (IL-17) is an essential proinflammatory cytokine, which is mainly secreted by the CD4+ helper T cells (Th17 cells) and subsets of innate lymphoid cells. IL-17A is associated with the pathogenesis of inflammatory diseases, including psoriasis, atopic dermatitis, hidradenitis suppurativa, alopecia areata, pityriasis rubra pilaris, pemphigus, and systemic sclerosis. Interleukin-23 (IL-23) is a member of the IL-12 family of cytokines with pro-inflammatory properties. IL-23 plays a pivotal role in stimulating the production of IL-17 by activating the Th17 cells. The IL-23/IL-17 axis is an important pathway for targeted therapy for inflammatory diseases. Its ability to potently enhance the expansion of T helper type 17 (Th17) cells indicates the responsibility for many of the inflammatory autoimmune responses. Emerging data demonstrate that IL-23 is a key participant in central regulation of the cellular mechanisms involved in inflammation. IL-21 is a type I cytokine produced by T cells and natural killer T cells that has pleiotropic actions on a wide range of immune and non-immune cell types. Interleukin (IL)-33 is a new member of the IL-1 superfamily of cytokines that is expressed by mainly stromal cells, such as epithelial and endothelial cells, and its expression is upregulated following pro-inflammatory stimulation. IL-33 can function both as a traditional cytokine and as a nuclear factor regulating gene transcription. Interleukin-31, produced mainly by activated CD4+ T cells, is a newly discovered member of the gp130/IL-6 cytokine family. Binding of IL-31 to its receptor activates JAK/STAT, PI3K/AKT and MAPK pathways. IL-2 and IL-6 are the typical histamine-dependent mediators of pruritus. The indirect path of itch induction as mentioned above is dependent on the disruption of the epidermal barrier. This is usually accompanied by the production of pro-inflammatory cytokines (e.g. IL-6) and chemokines, e.g. CXCL-8, CCL17/TARC, CCL19/MIP-30, CCL22/MDC, CCL23/MIP-3, CCL4/MIP-1l and CXCL1/GRO1α.

IL-23 and IL-17 are elevated in pruritus-related skin disorders. The pathogenesis of Atopic Dermatitis (AD) involves epidermal barrier dysfunction, skin microbiome abnormalities, and cutaneous inflammation. Inflammatory mediators, such as interleukin (IL)-4, IL-13, IL-17, IL-23, IL-31, IL-33, and thymic stromal lymphopoietin, are involved in AD development. Allergic contact dermatitis is a delayed, type 4 hypersensitivity reaction, i.e., an antigen-specific T-cell mediated inflammatory response to repeated exposure. Irritant contact dermatitis is caused by chronic exposure to irritants that causes epidermal barrier perturbation.

Oclacitinib maleate (APOQUEL®) is the current standard for treatment of pruritus, in particular control of pruritus associated with allergic dermatitis and control of atopic dermatitis in dogs at least 12 months of age. The oral dose of APOQUEL tablets is 0.18 to 0.27 mg oclacitinib/lb (0.4 to 0.6 mg oclacitinib/kg) bodyweight, administered orally, twice daily for up to 14 days, and then administered once daily for maintenance therapy. APOQUEL may be administered with or without food. The effectiveness of oclacitinib against canine atopic dermatitis has been found to be dependent upon dose and mode of administration (topical versus oral). APOQUEL is a synthetic Janus Kinase (JAK) inhibitor, which reportedly inhibits the function of a variety of cytokines dependent on JAK enzyme activity. For oclacitinib, the target cytokines are those that are proinflammatory or have a role in allergic responses/pruritis, e.g. IL-31. Oclacitinib inhibits the function of JAK1-dependent cytokines involved in allergy and inflammation (IL-2, IL-4, IL-6, and IL-13) as well as pruritus (IL-31) at IC50's ranging from 36 to 249 nM. Oclacitinib has minimal effects on cytokines that do not activate the JAK1 enzyme in cells (erythropoietin, granulocyte/macrophage colony-stimulating factor, IL-12, IL-23; IC50's >1000 nM). Oclacitinib reduces the frequency of IL-4- and IL-10-, but not IFN-γ-producing murine CD4+ and CD8+ T cells, and it counteracts the induction of Type 1 Regulatory T Cells. OCL exerts a suppressive effect on Th2- but not Th1-mediated immunity. Oclacitinib may also exert undesirable effects on other cytokines (for example, those involved in host defense or hematopoiesis) with the potential for unwanted effects. The package label for the product includes the following warnings.
APOQUEL is not for use in dogs less than 12 months of age.
APOQUEL modulates the immune system.
APOQUEL is not for use in dogs with serious infections.
APOQUEL may increase susceptibility to infection, including demodicosis, and exacerbation of neoplastic conditions.
New neoplastic conditions (benign and malignant) were observed in dogs treated with APOQUEL during clinical studies and have been reported in the post-approval period.

Part of APOQUEL's immune system modulation is apparently one of immunosuppression, since it increases susceptibility to infection and may even cause new neoplastic conditions. Post FDA approval, the following symptoms have been reported in order of decreasing frequency: vomiting, lethargy, anorexia, diarrhea, elevated liver enzymes, dermatitis (i.e. crusts, pododermatitis, pyoderma), seizures, polydipsia, and demodicosis. Benign, malignant, and unclassified neoplasms, dermal masses (including papillomas and histiocytomas), lymphoma and other cancers have been reported. However, the immunosuppressive nature of oclacitinib appears to occur at doses higher than those required to treat allergic pruritus in dogs. It would be a significant advancement in the area of oclacitinib therapy to develop a cotherapeutic regimen with a drug that boosts, rather than suppresses, the innate immune system sufficiently to overcome the undesirable immunosuppression caused by oclacitinib. It would be an even more significant advancement if such cotherapeutic regimen were to provide even better treatment and prevention of pruritus.

Another commercially available product for treating atopic dermatitis, allergic dermatitis, or pruritus is CYTOPOINT™ (from Zoetis; www.cytopoint.com/dosing/dosing-and-storage), which is a caninized monoclonal antibody (C-mAb) treatment for dogs that specifically targets and neutralizes canine IL-31, reportedly with minimal impact on normal immune functions. It is administered monthly or bimonthly as a sustained release injection at a starting dose of about 2 mg/Kg of bodyweight that reportedly lasts 4-8 weeks. The product is available in single-use 1 mL vials containing 10 mg, 20 mg, 30 mg, or 40 mg of C-mAB per vial.

Nerium oleander, a member of the Nerium species, is an ornamental plant widely distributed in subtropical Asia, the southwestern United States, and the Mediterranean. Its medical and toxicological properties have long been recognized. In humans, it has been proposed for use, for example, in the treatment of hemorrhoids, ulcers, leprosy, snake bites, cancers, tumors, neurological disorders, warts, and cell-proliferative diseases. Zibbu et al. (J. Chem. Pharm. Res. (2010), 2(6), 351-358) provide a brief review on the chemistry and pharmacological activity of Nerium oleander.

Oleandrin is a cardiac glycoside obtained by extraction from Nerium oleander (Nerium odorum) plant. It is widely recognized in the animal industry that consumption of the plant material is toxic to animals and on occasion may result in fatal poisoning. (Rubini et al., “A probable fatal case of oleander (Nerium oleander) poisoning on a cattle farm: a new method of detection and quantitation of the oleandrin toxin in rumen” in Toxins (2019), 11, 442; Ceci et al., “Outbreak of oleander (Nerium oleander) poisoning in dairy cattle: clinical and food safety implications” in Toxins (2020), 12, 471; Aslani et al., “Clinical and pathological aspects of experimental oleander (Nerium oleander) toxicosis in sheep” in Vet. Res. Commun. (2004), 28, 609-616; Barbosa et al., “Toxicity in goats caused by oleander (Nerium oleander)” in Res. Vet. Sci. (2008), 85, 279-281; Soto-Blanco et al., “Acute cattle intoxication from Nerium oleander pods” in Trop. Anim. Health Prod. (2006), 38, 451-454).

Oleander is considered the most important cause of livestock poisoning in South Africa. Accidental intoxications have been reported in horses, donkeys, cattle, camelids (alpaca and llama), dogs, cats and pet birds. Mydriasis in animals, after oleander ingestion, is also observed in relation to the increased sympathetic tone. For this reason, no therapeutic products derived from the plant have been developed for use in animals such as commercial animals or livestock, e.g. horses, cows, pigs, goats, sheep, poultry, etc.

Extraction of components from plants of Nerium species has traditionally been carried out using boiling water, cold water, supercritical fluid, or organic solvent.

ANVIRZEL™ (U.S. Pat. No. 5,135,745 to Ozel) contains the concentrated form or powdered form of the hot-water extract of Nerium oleander. Muller et al. (Pharmazie. (1991) September 46(9), 657-663) disclose the results regarding the analysis of a water extract of Nerium oleander. They report that the polysaccharide present is primarily galacturonic acid. Other saccharides include rhamnose, arabinose and galactose. Polysaccharide content and individual sugar composition of polysaccharides within the hot water extract of Nerium oleander have also been reported by Newman et al. (J. Herbal Pharmacotherapy, (2001) vol 1, pp. 1-16). Compositional analysis of ANVIRZEL™, the hot water extract, was described by Newman et al. (Anal. Chem. (2000), 72(15), 3547-3552). U.S. Pat. No. 5,869,060 to Selvaraj et al. pertains to extracts of Nerium species and methods of production. To prepare the extract, plant material is placed in water and boiled. The crude extract is then separated from the plant matter and sterilized by filtration. The resultant extract can then be lyophilized to produce a powder. U.S. Pat. No. 6,565,897 (U.S. Pregrant Publication No. 20020114852 and PCT International Publication No. WO 2000/016793 to Selvaraj et al.) discloses a hot-water extraction process for the preparation of a substantially sterile water extract. Ishikawa et al. (J. Nutr. Sci. Vitaminol. (2007), 53, 166-173) discloses a hot water extract of Nerium oleander and fractionation thereof by liquid chromatography using mixtures of chloroform, methanol, and water. They also report that extracts of the leaves of N. oleander have been used to treat Type II diabetes. US20060188585 published Aug. 24, 2006 to Panyosan discloses a hot water extract of Nerium oleander. U.S. Pat. No. 10,323,055 issued Jun. 18, 2019 to Smothers discloses a method of extracting plant material with aloe and water to provide an extract comprising aloe and cardiac glycoside. US20070154573 published Jul. 5, 2007 to Rashan et al. discloses a cold-water extract of Nerium oleander and its use.

Erdemoglu et al. (J. Ethnopharmacol. (2003) November 89(1), 123-129) discloses results for the comparison of aqueous and ethanolic extracts of plants, including Nerium oleander, based upon their anti-nociceptive and anti-inflammatory activities. Fartyal et al. (J. Sci. Innov. Res. (2014), 3(4), 426-432) discloses results for the comparison of methanol, aqueous, and petroleum ether extracts of Nerium oleander based upon their antibacterial activity.

Organic solvent extracts of Nerium oleander are also disclosed by Adome et al. (Aft. Health Sci. (2003) August 3(2), 77-86; ethanolic extract), el-Shazly et al. (J. Egypt Soc. Parasitol. (1996), August 26(2), 461-473; ethanolic extract), Begum et al. (Phytochemistry (1999) February 50(3), 435-438; methanolic extract), Zia et al. (J. Ethnolpharmacol. (1995) November 49(1), 33-39; methanolic extract), and Vlasenko et al. (Farmatsiia. (1972) September-October 21(5), 46-47; alcoholic extract). Turkmen et al. (J. Planar Chroma. (2013), 26(3), 279-283) discloses an aqueous ethanol extract of Nerium oleander leaves and stems. U.S. Pat. No. 3,833,472 issued Sep. 3, 1974 to Yamauchi discloses extraction of Nerium odorum SOL (Nerium oleander Linn) leaves with water, organic solvent, or aqueous organic solvent, wherein the leaves are heated to 60°−170° C. and then extracted, and the organic solvent is methanol, ethanol, propyl ether or chloroform.

A supercritical fluid extract (PBI-05204) of Nerium species is known (U.S. Pat. Nos. 8,394,434, 8,187,644, 7,402,325) and has demonstrated efficacy in treating neurological disorders (U.S. Pat. Nos. 8,481,086, 9,220,778, 9,358,293, US 20160243143A1, U.S. Pat. Nos. 9,877,979, 10,383,886) and cell-proliferative disorders (U.S. Pat. Nos. 8,367,363, 9,494,589, 9,846,156), and some viral infections (U.S. Pat. No. 10,596,186, WO 2018053123A1, WO2019055119A1)

Triterpenes are known to possess a wide variety of therapeutic activities. Some of the known triterpenes include oleanolic acid, ursolic acid, betulinic acid, bardoxolone, maslinic acid, and others. The therapeutic activity of the triterpenes has primarily been evaluated individually rather than as combinations of triterpenes.

Compositions containing triterpenes in combination with other therapeutic components are found as plant extracts. Fumiko et al. (Biol. Pharm. Bull (2002), 25(11), 1485-1487) discloses the evaluation of a methanolic extract of Rosmarimus officinalis L. for treating trypanosomiasis. Addington et al. (U.S. Pat. Nos. 8,481,086, 9,220,778, 9,358,293, US 20160243143 A1) disclose a supercritical fluid extract (SCF; PBI-05204) of Nerium oleander containing oleandrin and triterpenes for the treatment of neurological conditions. Addington et al. (U.S. Pat. No. 9,011,937, US 20150283191 A1) disclose a triterpene-containing fraction (PBI-04711) of the SCF extract of Nerium oleander containing oleandrin and triterpenes for the treatment of neurological conditions. Jager et al. (Molecules (2009), 14, 2016-2031) disclose various plant extracts containing mixtures of oleanolic acid, ursolic acid, betulinic acid and other components. Mishra et al. (PLoS One 2016 25; 11(7):e0159430. Epub 2016 Jul. 25) disclose an extract of Betula utilis bark containing a mixture of oleanolic acid, ursolic acid, betulinic acid and other components. Wang et al. (Molecules (2016), 21, 139) disclose an extract of Alstonia scholaris containing a mixture of oleanolic acid, ursolic acid, betulinic acid and other components. L. e Silva et al. (Molecules (2012), 17, 12197) disclose an extract of Eriope blanchetti containing a mixture of oleanolic acid, ursolic acid, betulinic acid and other components. Rui et al. (Int. J. Mol. Sci. (2012), 13, 7648-7662) disclose an extract of Eucalyptus globulus containing a mixture of oleanolic acid, ursolic acid, betulinic acid and other components. Ayatollahi et al. (Iran. J. Pharm. Res. (2011), 10(2), 287-294) disclose an extract of Euphorbia microsciadia containing a mixture of oleanolic acid, ursolic acid, betulinic acid and other components. Wu et al. (Molecules (2011), 16, 1-15) disclose an extract of Ligustrum species containing a mixture of oleanolic acid, ursolic acid, betulinic acid and other components. Lee et al. (Biol. Pharm. Bull (2010), 33(2), 330) disclose an extract of Forsythia viridissima containing a mixture of oleanolic acid, ursolic acid, betulinic acid and other components.

Oleanolic acid (O or OA), ursolic acid (U or UA) and betulinic acid (B or BA) are the three major triterpene components found in PBI-05204 (PBI-23; a supercritical fluid extract of Nerium oleander) and PBI-04711 (a triterpene-containing fraction 0-4 of PBI-05204). Van Kanegan et al. previously reported (Nature Scientific Reports (May 2016), 6:25626. doi: 10.1038/srep25626) on the contribution of the triterpenes toward efficacy by comparing their neuroprotective activity in a brain slice oxygen glucose deprivation (OGD) model assay at similar concentrations. PBI-05204 (PBI) and PBI-04711 (Fraction 0-4) were found to provide neuroprotective activity.

Extracts of Nerium species are known to contain many different classes of compounds: cardiac glycosides, glycones, steroids, triterpenes, polysaccharides and others. Specific compounds include oleandrin, neritaloside, odoroside, oleanolic acid, ursolic acid, betulinic acid, oleandrigenin, desacetyloleandrin, gitoxigenin, oleaside A, betulin (urs-12-ene-3β,28-diol), 28-norurs-12-en-30-ol, urs-12-en-3β-ol, 3β,3β-hydroxy-12-oleanen-28-oic acid, 3β,20α-dihydroxyurs-21-en-28-oic acid, 3β,27-dihydroxy-12-ursen-28-oic acid, 3β,13β-dihydroxyurs-11-en-28-oic acid, 3β,12α-dihydroxyoleanan-28,13β-olide, 3β,27-dihydroxy-12-oleanan-28-oic acid, and other components.

The effect of aqueous leaf extract of Nerium oleander (80% and 100% applied twice daily) on healing of wound was investigated in incision wounds in rabbits. Wound showed complete healing at 6-7 days compared with local antibiotic fucine ointment without complication. The wound healing effects of Aloe vera-based extract of the Nerium oleander leaf (NAE-8®) (topical application twice a day for 14 consecutive days) were studied in thermal injury in comparison with silver sulfadiazine treatment in rats. Thermal injury-induced alterations in malondialdehyde, glutathione, myeloperoxidase, TNF-α, interleukin-1l, and % DNA in the tail, these alterations were significantly reversed by NAE-8® treatment. The ameliorative effects of NAE-8® were also supported by histological findings. The NAE-8© provided significantly better antioxidant protection in the cellular antioxidant protection of erythrocytes (CAP-e) bioassay than aqueous extract of Nerium oleander. NAE-8® and aqueous extract of Nerium oleander both protected cellular viability and intracellular reduced glutathione and reduced the ROS formation significantly when compared to control cells, both under inflamed and neutral culture conditions. The treatment of dermal fibroblasts with NAE-8® resulted in selective secretion of cytokines involved in collagen and hyaluronan production as well as re-epithelialization during wound healing. The authors concluded that NAE-8® showed beneficial antioxidant protection in several cellular models without the induction of leukocyte activation and secretion of inflammatory cytokines. (Benson et al. “Water soluble egg membrane enhances the immunoactivating properties of an aloe vera-based extract of Nerium oleander leaves” in Clin. Cosm. Invest. Derm. (2016), 9, 393-403, doi.org/10.2147/CCID.S114471; Benson et al. “Antioxidant, anti-inflammatory, anti-apoptotic, and skin regenerative properties of an aloe vera-based extract of Nerium oleander leaves (NAE-8)” in Clin. Cosm. Invest. Derm. (2015), 8, 239-248, doi.org/10.2147/CCID.S79871). The NAE-8 is prepared by extraction of Nerium oleander biomass with aloe vera gel, e.g. has been prepared according to the process described in U.S. Pat. No. 10,323,055 B2, which issued Jun. 18, 2019 and which was filed Jul. 17, 2013 as U.S. Ser. No. 13/944,720, U.S. Pat. No. 8,524,286 B2, which issued Sep. 3, 2013 and which was filed Oct. 13, 2009 as U.S. Ser. No. 12/578,436, and WO 2010045243 A1, which published Apr. 22, 2010.

On the other hand, numerous publications report the incidence of side effects associated with contact of Nerium oleander extract with skin. Nerium oleander extract is included in the Homeopathic Pharmacopoeia of the United States (HPUS). Riley (“Oleander” in Materia Medica of New and Old Homeopathic Medicines (2012), pp 147-148; Springer, Berlin, Heidelberg. https://doi.org/10.1007/978-3-642-25292-1_50) discusses the provings for (symptoms associated with consumption of) the HPUS oleander extract. Riley states, “This proving was also characterized by a large number of skin complaints. Many of these skin symptoms were of a dry and itchy nature, with flaking, scaly, and patchy eruptions also present. There was a tendency for the skin symptoms to be visible and red. These skin symptoms occurred on the head, the back, and the extremities.” In other words, Riley specifically teaches that administration of oleander extract causes pruritus.

Tailor et al. (“Oleander-associated keratitis and uveitis” in Cornea (2022), 41(10), 1305-1307) report the occurrence of keratitis in a subject having contacted plant material. As well, Apted (“Oleander dermatitis” in Contact Dermatitis (1983), 9, 321-321, https://doi.org/10.1111/j.1600-0536.1983.tb04406.x) reports that dermatitis has frequently been reported upon contact with oleander plant material.

Therefore, it would be extremely unexpected that Nerium oleander extract or oleandrin would provide any improvement in pruritus or itch-related skin disorders, in particular dermatitis or pruritus.

A need remains for compositions containing oleandrin, optionally together with one or more other compounds that are effective against specific itch-related (pruritus-related) skin disorders in animals and humans. A need also remains for a cotherapeutic regimen that improves the antipruritic effectiveness of oclacitinib while concomitantly reducing its undesirable immunosuppressive activity.

### SUMMARY OF THE INVENTION

The invention provides a pharmaceutical composition and method for treating and/or preventing itch-related skin disorders (pruritus-related disorders) in animals or humans. The invention also provides a method of treating and/or preventing an itch-related skin disorder in animals and humans by non-systemic or systemic administration of the pharmaceutical composition. The inventors have succeeded in preparing compositions that exhibit sufficient anti-pruritic activity to justify their use in treating pruritus or itch-related skin disorders, while at the same time being administered at doses that are not non-toxic (completely safe) to the animals or humans. The inventors have developed corresponding treatment and prevention methods employing particular dosing regimens.

The inventors have also developed a cotherapeutic regimen for treating pruritus or itch-related disorders, e.g. atopic dermatitis or allergic dermatitis, particularly in canines, said cotherapeutic regimen including treating a subject with at least a) oleandrin-containing composition and oclacitinib-containing composition; or b) oleandrin-containing composition and caninized monoclonal antibody (C-mAb) treatment for dogs that specifically targets and neutralizes canine IL-31; wherein the compositions can be administered at the same time or separately.

The methods of the invention comprise chronically administering to a subject in need thereof one or more doses of oleandrin-containing composition (OCC) on a recurring basis over a treatment period, thereby treating and/or preventing the itch-related skin disorder in the subject. In some embodiments, the itch-related skin disorder is selected from the group consisting of pruritus, urticaria, xerosis (dry skin), eczema (atopic dermatitis), psoriasis, hives, welts, and nettle rash. The pruritus may be associated with allergic contact dermatitis (caused by allergy to allergens, e.g. fleas, dust mites, grass, plant pollens, chemicals, etc.), irritant contact dermatitis, food hypersensitivity dermatitis, or allergy to medication. In some embodiments, the itch-related skin disorder is not caused by a pathogen, e.g. bacteria, virus, or fungus. In some embodiments, the pruritic skin does not have any open wounds, blisters, deep cuts, or severe burns.

In every embodiment of the invention, the OCC may also be referred to as a cardiac glycoside-containing composition (CGCC). Oleandrin and digoxin are both cardiac glycosides. Unless otherwise specified herein, the CGCC comprises a) oleandrin (excluding digoxin); b) digoxin (excluding oleandrin); or c) a combination of oleandrin and digoxin. DCC refers to a digoxin-containing composition.

Pruritus affecting cats is referred to as “atopic-like dermatitis”, “nonflea, nonfood hypersensitivity dermatitis” (NFNFHD), or “feline atopic skin syndrome” (FASS). The OCC and respective methods of the invention are suitable for treating and preventing such feline disorders.

Administration of the OCC can be a) one or more times daily; b) at least three days per week; c) at least five days per week; and/or d) every day of the week. A treatment period can last at least three days, at least five days, at least one week, at least 10 days, at least two weeks, at least three weeks, at least one month, at least two months, or long. One or more doses are administered on a daily, weekly, and/or monthly basis. One or more doses per day can be administered. One, two, three, four, five, six or more doses can be administered per day. The composition can be administered chronically, i.e. on a recurring basis, such as daily, every other day, every second day, every third day, every fourth day, every fifth day, every sixth day, weekly, every other week, every second week, every third week, monthly, bimonthly, semi-monthly, every other month every second month, quarterly, every other quarter, trimesterly, seasonally, semi-annually and/or annually. The treatment period may be for one or more weeks, one or more months, one or more quarters and/or one or more years. An effective dose of oleandrin-containing composition can be administered one or more times a day.

Chronic administration can be achieved by repeated administration of dosage forms. Suitable oral dosage forms include rapid or immediate release dosage form(s), delayed release, and/or extended (controlled, sustained, prolonged) release dosage form(s). Suitable topical dosage forms include powder, liquid, gel, cream, lotion, paste, foam, solution, suspension, emulsion, ointment, or other such dosage forms. The composition is preferably administered topically.

The subject can be an animal or human. The animal can be a domestic or livestock animal, e.g. dog, cat, cow, horse, sheep, goat, pig, llama, alpaca, buffalo, deer, elk, giraffe, camel, or other hair-bearing or fur-bearing animal. In some embodiments, the animal is further afflicted with malignant neoplasia, cancer, tumor, or immune suppression. In some embodiments, the animal is also undergoing therapy with one or more drugs that suppress the immune system.

An aspect of the invention provides an antipruritic composition comprising a) oleandrin; and b) one or more pharmaceutical excipients. Another aspect of the invention provides an anti-pruritic comprising oleandrin, one or more pharmaceutically acceptable (for animal or human use) excipients, and at least one other anti-pruritic component.

In some embodiments, the composition further comprises one or more of the following: a) one or more compounds extracted from Nerium oleander plant material; b) one or more triterpenoic acids, e.g. one or more of oleanolic acid, ursolic acid, or betulinic acid; c) one or more topical anesthetics; d) one or more emollients; e) one or more antioxidants; f) one or more solvents; g) one or more oils; h) allantoin; i) oclacitinib, optionally present as the maleate salt (APOQUEL®); j) one or more active polysaccharides from aloe vera, said polysaccharides being selected from the group consisting of glucomannan, polymannose, acemannan, and 1-4)-acetylated polymannose; k) colloidal oatmeal; l) one or more preservatives; m) one or more surfactants; n) one or more anti-foaming agents; o) one or more lubricants; p) one or more corticosteroids (glucocorticoids); q) collagen, e.g. hydrolyzed collagen; r) one or more carboxylic acids; s) one or more moisturizers (hydrating agents); t) one or more amino acids; u) one or more humectants; v) one or more buffering agents; w) one or more topical skin cleansers; x) one or more antihistamines; y) cyclosporine; z) one or more skin permeation enhancers; aa) epigallocatechin-3-gallate (EGCG); bb) green tea extract; cc) polyphenol. Embodiments of the invention include those wherein one or more compounds in the composition is/are present in free base form, free acid form, and/or salt form. Some of the above components also possess anti-pruritic activity; and/or dd) caninized monoclonal antibody (C-mAb) treatment for dogs that specifically targets and neutralizes canine IL-31.

In some embodiments, a) the one or more oils is selected from the group consisting of bitter orange oil, chamomile oil, clove oil, silicone oil (dimethicone; polydimethylsiloxane), and others; b) the one or more solvents is selected from the group consisting of water, caprylyl glycol, hexylene glycol, phenoxyethanol, ethanol, glycerin (glycerol), propylene glycol, and others; c) the one or more antioxidants is selected from the group consisting of tocopherol (vitamin E), and others; d) the one or more topical anesthetics is selected from the group consisting of pramoxine, lidocaine, prilocaine, and others; and/or e) the one or more carboxylic acids is selected from the group consisting of pyrrolidine carboxylic acid (PCA), ascorbic acid, citric acid, acetic acid, salicylic acid, and others.

Another aspect of the invention provides a cotherapeutic method of treating or preventing pruritis, in particular allergic dermatitis and atopic dermatitis, in dogs, the method comprising administering to a dog one or more doses of OCC and one or more doses of oclacitinib (or salt thereof)-containing composition. The oleandrin and oclacitinib maybe administered a) in the same composition or in separate compositions; b) via the same route of administration or via different routes of administration; c) simultaneously, sequentially, or in an overlapping manner; and/or d) with overlapping dosing periods or separate dosing periods.

The established doses of oclacitinib are suitable for use according to the invention. The oral dose of APOQUEL (oclacitinib maleate) tablets is 0.18 to 0.27 mg oclacitinib/lb (0.4 to 0.6 mg oclacitinib/kg) bodyweight, administered orally, twice daily for up to 14 days, and then administered once daily for maintenance therapy. In some embodiments, the dose of oclacitinib is reduced because of the additive or synergistic anti-pruritic activity of cardiac glycoside, in particular oleandrin.

In some embodiments, a) the OCC and oclacitinib-containing composition are both administered systemically; b) the OCC is administered non-systemically, and the oclacitinib-containing composition is administered systemically; c) the OCC is administered systemically, and the oclacitinib-containing composition is administered non-systemically; d) the OCC is administered orally, buccally, sublingually, and/or topically, and the oclacitinib-containing composition is administered orally, buccally, sublingually, and/or topically.

Another aspect of the invention provides a cotherapeutic method of treating or preventing pruritis, in particular allergic dermatitis and atopic dermatitis, in dogs, the method comprising administering to a dog one or more doses of OCC and one or more doses of C-mAb-containing composition. The oleandrin and C-mAb maybe administered a) in the same composition or in separate compositions; b) via the same route of administration or via different routes of administration; c) simultaneously, sequentially, or in an overlapping manner; and/or d) with overlapping dosing periods or separate dosing periods.

The established doses of oclacitinib are suitable for use according to the invention. The injectable dose of C-mAb is 2 mg/Kg bodyweight, administered monthly or bimonthly by injection monthly for breakthrough or maintenance therapy. Higher doses may be required. In some embodiments, the dose of C-mAb is reduced because of the additive or synergistic anti-pruritic activity of cardiac glycoside, in particular oleandrin.

In some embodiments, a) the OCC and C-mAb-containing composition are both administered systemically; b) the OCC is administered non-systemically, and the C-mAb-containing composition is administered systemically; c) the OCC is administered systemically, and the C-mAb-containing composition is administered non-systemically; d) the OCC is administered orally, buccally, sublingually, and/or topically, and the C-mAb-containing composition is administered orally, buccally, sublingually, and/or topically.

The invention also provides an anti-pruritic composition comprising cardiac glycoside, e.g. oleandrin or digoxin, and C-mAB. One or more excipients or other compounds as described herein can be further included in the anti-pruritic composition.

The invention also provides an anti-pruritic composition comprising cardiac glycoside, e.g. oleandrin or digoxin, and oclacitinib (or salt thereof). One or more excipients or other compounds as described herein can be further included in the anti-pruritic composition.

Another aspect of the invention provides a method of preventing an animal from exhibiting one or more symptoms associated with pruritus, the method comprising administering to said animal one or more effective doses of oleandrin-containing composition (OCC). The method optionally further comprises administering oclacitinib-containing compositions or C-mAB-containing compositions. The composition(s) may be administered to prevent occurrence or recurrence of pruritus in the animal. For example, the composition(s) may be administered prior to the animal exhibiting symptoms of pruritus (prevention of occurrence) or as a maintenance therapy after pruritus in the animal has already been resolved (prevention of recurrence).

Administration of one or more effective doses of an oleandrin-containing composition to a human or animal subject may help a) maintain healthy skin; b) maintain healthy coat in an animal; c) support coat health in an animal; d) support skin health in an animal; e) support a healthy response to seasonal skin allergy; f) support the immune system; g) assist the immune system; h) enhance the immune response; i) provide immune enhancing effects; j) provide nutritional support for healthy immune system; k) support a healthy immune system; l) support immune system function; m) support and promote long-term health; n) promote the body's innate resistance to pathogens; o) reduce oxidative stress; p) provide antioxidant support; q) help maintain a normal inflammatory response; and/or r) maintain a normal inflammatory cytokine cascade. The OCC can be administered with oclacitinib-containing composition or C-mAB-containing composition.

The equivalent of plural daily doses of oleandrin can be achieved by administering to said animal one or more extended-release dosage forms that release therapeutically effective daily doses of oleandrin throughout a treatment period. Additional means of administering effective daily doses may be achieved through use of dosage forms suitable for use in water, milk, liquid feed, milk substitute, colostrum, colostrum substitute, edible treat, or solid feed.

The invention also provides a method of treating pruritus in an animal, the method comprising administering to the animal 1-10 doses of oleandrin-containing composition per day for a treatment period of 2 days to about 2 months. Two to eight, two to six, or four doses can be administered daily during the treatment period. Doses can be administered for 2 days to about 60 days, 2 days to about 45 days, 2 days to about 30 days, 2 days to about 21 days, or 2 days to about 14 days. Said administering can be through any of the modes of administration discussed herein.

An oleandrin-containing composition comprises oleandrin, which may be provided in pure form or as part of an extract of Nerium sp. plant material(s), wherein the extract further comprises one or more components extracted from said plant material(s).

In some embodiments, an oral composition further comprises at least one cardiac glycoside-metabolism inhibitor, at least one cardiac glycoside-digestion inhibitor, at least one enzyme inhibitor, or a combination thereof.

A veterinary clinician will be able to use known dose escalation or de-escalation protocols to determine a safe and effective dose of oleandrin to be administered to a subject. The dose or dosing regimen can be adjusted as needed until the subject reaches the desired clinical endpoint(s) such as a reduction, alleviation, or elimination specific symptoms associated with pruritus, e.g. repeated scratching and/or licking of itchy skin.

The oral maximum tolerated dose (MTD) of oleandrin may vary according to animal species. In some embodiments, a) said animal is a cow and the dose of composition provides a maximum plasma concentration of oleandrin of no more than 1 ng/mL; b) said animal is a pig and the dose of composition provides a maximum plasma concentration of oleandrin of no more than 5 ng/mL; c) said animal is a horse and the dose of composition provides a maximum plasma concentration of or oleandrin of no more than 5 ng/mL; d) said animal is a sheep and the dose of composition provides a maximum plasma concentration of oleandrin of no more than 5 ng/mL; or e) said animal is a goat and the dose of composition provides a maximum plasma concentration of oleandrin of no more than 10 ng/mL.

Suitable target oral doses (one to four times daily) for oleandrin in animals are as follows: a) in dogs—less than about 100 microg/kg bodyweight, about 0.5-60 microg/kg bodyweight, about 0.1-10 microg/kg bodyweight, or about 0.1-5 microg/kg bodyweight; b) in cows—less than about 100 micro/kg bodyweight or about 5-50 microg/kg bodyweight; c) in cats—less than about 100 microg/kg bodyweight, about 0.5-50 microg/kg bodyweight, about 0.1-10 microg/kg bodyweight, or about 0.1-5 microg/kg bodyweight; and d) in horses—less than about 100 microg/kg bodyweight or 0.5-50 microg/kg bodyweight.

Where oleandrin is administered in the form of Nerium species (Nerium sp.), e.g. Nerium oleander or Nerium indicum, leaf material, the amount of dried leaf material will preferably be a) less than 100 mg/Kg bodyweight or less than 50 mg/Kg bodyweight for a cow; b) less than 110 mg/Kg bodyweight for a goat; c) less than 110 mg/Kg bodyweight or less than 250 mg/Kg bodyweight for a sheep.

In some embodiments, following oral administration, the concentration of oleandrin in the plasma of a treated subject is about 10 ng/mL or less, about 5 ng/mL or less, about 2.5 ng/mL or less, about 2 ng/mL or less, about 1 ng/mL, or about 0.5 ng/mL or less. In some embodiments, following oral administration, the concentration of oleandrin in the plasma of a treated animal is about 0.0001 ng/mL or more, about 0.0005 ng/mL or more, about 0.001 ng/mL or more, about 0.0015 ng/mL or more, about 0.01 ng/mL or more, about 0.015 ng/mL or more, about 0.1 ng/mL or more, about 0.15 ng/mL or more, about 0.05 ng/mL or more, or about 0.075 ng/mL or more. The daily oral dose of composition administered will be sufficient to provide a plasma concentration of oleandrin within at least one of the ranges set forth herein. The invention includes all combinations and selections of the plasma concentration ranges set forth herein.

The dose of oleandrin can also be about 0.5 to about 500 microg/day or less, about 0.5 to about 400 microg/day or less, about 0.5 to about 300 microg/day or less, about 0.5 to about 200 microg/day or less, about 0.5 to about 100 microg/day or less, about 1 to about 80 microg/day, about 1.5 to about 60 microg/day, about 1.8 to about 60 microg/day, about 1.8 to about 40 microg/day.

All dosing regimens, dosing schedules, and doses described herein are contemplated as being suitable; however, some dosing regimens, dosing schedules, and doses may be more suitable for some subjects than for others. The target clinical endpoints are used to guide said dosing.

The composition can be administered systemically. Modes of systemic administration include parenteral, buccal, enteral, intramuscular, subdermal, sublingual, peroral, pulmonary, or oral. The composition can also be administered via injection or intravenously. The composition may also be administered by two or more routes to the same subject. In some embodiments, the composition is administered by a combination of any two or more modes of administration selected from the group consisting of parenteral, buccal, enteral, intramuscular, subdermal, sublingual, peroral, pulmonary, intranasal, and oral.

The oleandrin may also be included in a feed and/or a liquid and administered orally to the animal. The solid feed may comprise oleandrin and at least one feedstuff. The liquid feed may comprise oleandrin, at least one liquid, and at least one nutrient. Oleandrin may also be administered in a milk substitute product or in water. The oleandrin may also be administered to the animal by feeding the animal leaf material from the Nerium sp. plant. The leaf material may be dried or undried.

In some embodiments, the OCC is included in a topical composition, optionally excluding one or more skin permeation enhancers. In other embodiments, a topical composition includes one or more skin permeation enhancers.

The invention also provides a sublingual dosage form comprising oleandrin and liquid carrier. The invention also provides a method of treating pruritus comprising sublingually administering plural doses of an oleandrin-containing composition to an animal having pruritus. The invention also provides a method of preventing pruritus comprising sublingually administering plural doses of an oleandrin-containing composition to an animal prone to exhibiting symptoms of pruritus. One or more doses can be administered per day for two or more days per week and for one or more weeks per month, optionally for one or months per year. The liquid carrier can comprise water, oil, liquid feed, or a combination of any thereof.

In some embodiments, the composition comprises oleandrin and oil. The oil can comprise medium chain triglycerides (MCT). The composition can comprise one, two or more oleandrin-containing extracts and one or more pharmaceutical excipients.

In some embodiments, the oleandrin-containing composition comprises an extract of Nerium sp., said extract comprising a) at least oleandrin; b) at least oleandrin, oleanolic acid, ursolic acid, and betulinic acid; c) at least oleandrin, oleanolic acid, ursolic acid, betulinic acid, oleandrigenin, desacetyl-oleandrin, and gitoxigenin; d) at least oleandrin, oleanolic acid, ursolic acid, betulinic acid, oleandrigenin, desacetyl-oleandrin, gitoxigenin, kanerocin, and kanerodione; or e) at least at least oleandrin, oleanolic acid, ursolic acid, betulinic acid, oleandrigenin, gitoxigenin, desacetyl-oleandrin, kanerocin, kanerodione, Nerium F, neritaloside, odoroside, adynerin, and odoroside-G-acetate.

The oleandrin-containing composition (or the extract) may further comprise polyphenol(s), carbohydrate(s), flavonoid(s), amino acid(s), soluble protein(s), cellulose, starch, alkaloid(s), saponin(s), tannin(s), and any combination thereof.

The amino acid can be selected from the group consisting of aspartic acid, glutamic acid, asparagine, serine, glutamine, glycine, histidine, arginine, threonine, alanine, proline, tyrosine, valine, methionine, cysteine, isoleucine, leucine, phenylalanine, tryptophan, and lysine. In some embodiments, the amino is selected from the group consisting of asparagine, arginine, threonine, alanine, proline, tyrosine, valine, isoleucine, leucine, phenylalanine, tryptophan, and lysine.

The aglycone oleandrigenin can also be further included. In some embodiments, the composition further comprises a) one or more triterpenes; b) one or more steroids; c) one or more triterpene derivatives; d) one or more steroid derivatives; or e) a combination thereof. In some embodiments, the composition comprises oleandrin and a) two or three triterpenes; b) two or three triterpene derivatives; c) two or three triterpene salts; or d) a combination thereof. In some embodiments, the triterpene is selected from the group consisting of oleanolic acid, ursolic acid, betulinic acid, and salts or derivatives thereof. As used herein, the generic term triterpene also encompasses salts and derivatives thereof, unless otherwise specified.

The oleandrin can be present in a pharmaceutical composition in pure form or as part of an extract containing oleandrin. In some embodiments, the oleandrin is present as the primary therapeutic component, meaning the component primarily responsible for antipruritic activity of the composition.

In some embodiments, an oleandrin-containing extract is obtained by extraction of plant material. The extract can comprise a hot-water extract, cold-water extract, supercritical fluid (SCF) extract, subcritical liquid extract, organic solvent extract, or combination thereof of the plant material. In some embodiments, the extract has been (biomass) prepared by subcritical liquid extraction of Nerium plant mass (biomass) using, as the extraction fluid, subcritical liquid carbon dioxide, optionally comprising alcohol. In some embodiments, the oleandrin-containing composition comprises two or more different types of oleandrin-containing extracts.

Embodiments of the invention include those wherein the oleandrin-containing biomass (plant material) is Nerium sp., e.g. Nerium oleander, Nerium oleander L (Apocynaceae), Nerium odourum, Nerium indicum Mill, white oleander, pink oleander, Agrobacterium tumefaciens, cell culture (cellular mass) of any of said species, or a combination thereof. In some embodiments, the biomass comprises leaves, stems, flowers, bark, fruits, seeds, sap, and/or pods.

In some embodiments, the molar ratio of total triterpene content (OA+UA+BA) to oleandrin ranges from about 15:1 to about 5:1, or about 12:1 to about 8:1, or about 100:1 to about 15:1, or about 100:1 to about 50:1, or about 100:1 to about 75:1, or about 100:1 to about 80:1, or about 100:1 to about 90:1, or about 10:1.

In some embodiments, the molar ratios of the individual triterpenes to oleandrin range as follows: about 2-8 (OA):about 2-8 (UA):about 0.1-1 (BA):about 0.5-1.5 (OL); or about 3-6 (OA):about 3-6 (UA):about 0.3-8 (BA):about 0.7-1.2 (OL); or about 4-5 (OA):about 4-5 (UA):about 0.4-0.7 (BA):about 0.9-1.1 (OL); about 4.6 (OA):about 4.4 (UA):about 0.6 (BA):about 1 (OL); about 9-12: up to about 2: up to about 2, or about 10:about 1:about 1, or about 9-12:about 0.1-2:about 0.1-2, or about 9-11:about 0.5-1.5:about 0.5-1.5, or about 9.5-10.5:about 0.75-1.25:about 0.75-1.25, or about 9.5-10.5:about 0.8-1.2:about 0.8-1.2, or about 9.75-10.5:about 0.9-1.1:about 0.9-1.1.

In some embodiments, the other therapeutic agent, such as that obtained by extraction of Nerium sp. plant material, is not a polysaccharide obtained during preparation of the extract, meaning it is not an acidic homopolygalacturonan or arabinogalaturonan. In some embodiments, the extract excludes another therapeutic agent and/or excludes an acidic homopolygalacturonan or arabinogalaturonan obtained during preparation of the extract.

In some embodiments, the method excludes administration of an aloe extract of Nerium oleander and the composition excludes an aloe extract of Nerium oleander, wherein the extract has been prepared by extraction of Nerium oleander biomass with aloe vera gel, e.g. has been prepared according to the process described in U.S. Pat. No. 10,323,055 B2, which issued Jun. 18, 2019 and which was filed Jul. 17, 2013 as U.S. Ser. No. 13/944,720, U.S. Pat. No. 8,524,286 B2, which issued Sep. 3, 2013 and which was filed Oct. 13, 2009 as U.S. Ser. No. 12/578,436, and WO 2010045243 A1, which published Apr. 22, 2010. In some embodiments, the method excludes administration of egg membrane and of an aloe extract of Nerium oleander, and the composition excludes egg membrane and excludes the aloe extract of Nerium oleander, wherein the extract has been prepared by extraction of Nerium oleander biomass with aloe vera gel.

In some embodiments, the other therapeutic agent, such as that obtained by extraction of Nerium sp. plant material, is a polysaccharide obtained during preparation of the extract, e.g. an acidic homopolygalacturonan or arabinogalaturonan. In some embodiments, the extract comprises another therapeutic agent and/or comprises an acidic homopolygalacturonan or arabinogalaturonan obtained during preparation of the extract from said plant material.

In some embodiments, the extract comprises oleandrin and at least one other compound selected from the group consisting of glycone, aglycone, steroid, triterpene, polysaccharide, saccharide, alkaloid, fat, protein, neritaloside, odoroside, oleanolic acid, ursolic acid, betulinic acid, oleandrigenin, gitoxigenin, desacetyl-oleandrin, oleaside A, betulin (urs-12-ene-3β,28-diol), 28-norurs-12-en-3β-ol, urs-12-en-3β-ol, 3β,3β-hydroxy-12-oleanen-28-oic acid, 3β,20α-dihydroxyurs-21-en-28-oic acid, 3β,27-dihydroxy-12-ursen-28-oic acid, 3β,13β-dihydroxyurs-11-en-28-oic acid, 3β,12α-dihydroxyoleanan-28,13β-olide, 3β,27-dihydroxy-12-oleanan-28-oic acid, homopolygalacturonan, arabinogalaturonan, chlorogenic acid, caffeic acid, L-quinic acid, 4-coumaroyl-CoA, 3-O-caffeoylquinic acid, 5-O-caffeoylquinic acid, cardenolide B-1, cardenolide B-2, oleagenin, neridiginoside, nerizoside, odoroside-H, 3-beta-O-(D-diginosyl)-5-beta, 14 beta-dihydroxy-card-20(22)-enolide pectic polysaccharide composed of galacturonic acid, rhamnose, arabinose, xylose, and galactose, polysaccharide with MW in the range of 17000-120000 D, or MW about 35000 D, about 3000 D, about 5500 D, or about 12000 D, cardenolide monoglycoside, cardenolide N-1, cardenolide N-2, cardenolide N-3, cardenolide N-4, pregnane, 4,6-diene-3,12,20-trione, 20R-hydroxypregna-4,6-diene-3,12-dione, 16beta,17beta-epoxy-12beta-hydroxypregna-4,6-diene-3,20-dione, 12beta-hydroxypregna-4,6,16-triene-3,20-dione (neridienone A), 20S,21-dihydroxypregna-4,6-diene-3,12-dione (neridienone B), neriucoumaric acid, isoneriucoumaric acid, oleanderoic acid, oleanderen, 8alpha-methoxylabdan-18-oic acid, 12-ursene, kaneroside, neriumoside, 3β-O-(D-diginosyl)-2α-hydroxy-8,14β-epoxy-5β-carda-16:17, 20: 22-dienolide, 3β-O-(D-diginosyl)-2α,14β-dihydroxy-5β-carda-16:17,20:22-dienolide, 3β,27-dihydroxy-urs-18-en-13,28-olide, 3β,22α,28-trihydroxy-25-nor-lup-1(10),20(29)-dien-2-one, cis-karenin (3β-hydroxy-28-Z-p-coumaroyloxy-urs-12-en-27-oic acid), trans-karenin (3-β-hydroxy-28-E-p-coumaroyloxy-urs-12-en-27-oic acid), 3beta-hydroxy-5alpha-carda-14(15),20(22)-dienolide (beta-anhydroepidigitoxigenin), 3 beta-O-(D-digitalosyl)-21-hydroxy-5beta-carda-8,14,16,20(22)-tetraenolide (neriumogenin-A-3beta-D-digitaloside), proceragenin, neridienone A, 3beta,27-dihydroxy-12-ursen-28-oic acid, 3beta,13beta-dihydroxyurs-11-en-28-oic acid, 3beta-hydroxyurs-12-en-28-aldehyde, 28-orurs-12-en-3beta-ol, urs-12-en-3beta-ol, urs-12-ene-3beta,28-diol, 3beta,27-dihydroxy-12-oleanen-28-oic acid, (20S, 24R)-epoxydammarane-3beta,25-diol, 20beta,28-epoxy-28alpha-methoxytaraxasteran-3beta-ol, 20beta,28-epoxytaraxaster-21-en-3beta-ol, 28-nor-urs-12-ene-3beta,17 beta-diol, 3beta-hydroxyurs-12-en-28-aldehyde, alpha-neriursate, beta-neriursate, 3alpha-acetophenoxy-urs-12-en-28-oic acid, 3beta-acetophenoxy-urs-12-en-28-oic acid, oleanderolic acid, kanerodione, 3β-p-hydroxyphenoxy-11α-methoxy-12α-hydroxy-20-ursen-28-oic acid, 28-hydroxy-20(29)-lupen-3,7-dione, kanerocin, 3alpha-hydroxy-urs-18,20-dien-28-oic acid, D-sarmentose, D-diginose, neridiginoside, nerizoside, isoricinoleic acid, gentiobiosylnerigoside, gentiobiosylbeaumontoside, gentiobiosyloleandrin, folinerin, 12β-hydroxy-5β-carda-8,14,16,20(22)-tetraenolide, 8β-hydroxy-digitoxigenin, Δ16-8β-hydroxy-digitoxigenin, A16-neriagenin, uvaol, ursolic aldehyde, 27(p-coumaroyloxy)ursolic acid, oleanderol, 16-anhydro-deacteyl-nerigoside, 9-D-hydroxy-cis-12-octadecanoic acid, adigoside, adynerin, alpha-amyrin, beta-sitosterol, campestrol, caoutchouc, capric acid, caprylic acid, choline, cornerin, cortenerin, deacetyloleandrin, diacetyl-nerigoside, foliandrin, pseudocuramine, quercetin, quercetin-3-rhamnoglucoside, quercitrin, rosaginin, rutin, stearic acid, stigmasterol, strospeside, urehitoxin, and uzarigenin. Additional components that may be present in the extract are disclosed by Gupta et al. (IJPSR (2010), 1(3), 21-27, the entire disclosure of which is hereby incorporated by reference).

Oleandrin may also be obtained from extracts of suspension cultures derived from Agrobacterium tumefaciens-transformed calli (Ibrahim et al., “Stimulation of oleandrin production by combined Agrobacterium tumefaciens mediated transformation and fungal elicitation in Nerium oleander cell cultures” in Enz. Microbial Techno. (2007), 41(3), 331-336, the entire disclosure of which is hereby incorporated by reference). Hot water, organic solvent, aqueous organic solvent, subcritical liquid extract, or supercritical fluid extract of Agrobacterium may be used according to the invention.

Oleandrin may also be obtained from extracts of Nerium sp. microculture in vitro, whereby shoot cultures can be initiated from seedlings and/or from shoot apices of the Nerium sp. cultivars, e.g. Splendens Giganteum, Revanche or Alsace, or other cultivars (Vila et al., “Micropropagation of Oleander (Nerium oleander L.)” in HortScience (2010), 45(1), 98-102, the entire disclosure of which is hereby incorporated by reference). Hot water, organic solvent, aqueous organic solvent, or supercritical fluid extracts of microcultured Nerium sp. may be used according to the invention.

The extract may also be obtained by extraction of cellular mass (such as is present in cell culture) of any of said plant species.

The invention also provides use of OCC in the manufacture of a medicament for the treatment of pruritus in an animal or human. In some embodiments, the manufacture of such a medicament comprises: providing one or more compositions of the invention; including a dose of said composition in a pharmaceutical dosage form; and packaging the pharmaceutical dosage form. In some embodiments, the manufacture can be conducted as described in PCT International Application No. PCT/US06/29061. The manufacture can also include one or more additional steps such as: delivering the packaged dosage form to a vendor (retailer, wholesaler and/or distributor); selling or otherwise providing the packaged dosage form to an animal having pruritus; including with the medicament a label and a package insert, which provides instructions on use, dosing regimen, administration, content and toxicology profile of the dosage form. In some embodiments, the treatment of pruritus comprises: determining that a subject has pruritus; indicating administration of pharmaceutical dosage form to the subject according to a dosing regimen; administering to the subject one or more pharmaceutical dosage forms, wherein the one or more pharmaceutical dosage forms is administered according to the dosing regimen.

The pharmaceutical composition can further comprise a combination of at least one material selected from the group consisting of a water soluble (miscible) co-solvent, a water insoluble (immiscible) co-solvent, a surfactant, an antioxidant, a chelating agent, and an absorption enhancer.

The solubilizer is at least a single surfactant, but it can also be a combination of materials such as a combination of: a) surfactant and water miscible solvent; b) surfactant and water immiscible solvent; c) surfactant, antioxidant; d) surfactant, antioxidant, and water miscible solvent; e) surfactant, antioxidant, and water immiscible solvent; f) surfactant, water miscible solvent, and water immiscible solvent; or g) surfactant, antioxidant, water miscible solvent, and water immiscible solvent.

The pharmaceutical composition optionally further comprises a) at least one liquid carrier; b) at least one emulsifying agent; c) at least one solubilizing agent; d) at least one dispersing agent; e) at least one other excipient; or f) a combination thereof.

In some embodiments, the water miscible solvent is low molecular weight (less than 6000) PEG, glycol, or alcohol. In some embodiments, the surfactant is a pegylated surfactant, meaning a surfactant comprising a poly(ethylene glycol) functional group.

The invention includes all combinations of the aspects, embodiments and sub-embodiments of the invention disclosed herein.

### BRIEF DESCRIPTION OF THE FIGURES

The following figures form part of the present description and describe exemplary embodiments of the claimed invention. The skilled artisan will, in light of these figures and the description herein, be able to practice the invention without undue experimentation.

FIG. 1 depicts a chart comparing the relative impact of oleandrin (in vitro assay according to Example 13) versus oclacitinib (published data) upon particular cytokines.

FIG. 2 depicts a chart indicating the effect of oleandrin (in vitro assay according to Example 13) upon particular cytokines associated with the skin or skin disorders, such as pruritus.

FIG. 3 depicts a graph comparing the percent change of IL-23 levels, under inflamed culture conditions, following exposure of human PBMCs to oleandrin or an extract (PBI) containing oleandrin. The percent change is calculated based on a comparison to the mean fluorescence intensity of matching dose DMSO control cultures. Results are shown as the average±standard deviation of duplicate samples.

FIG. 4 depicts a chart comparing the relative impact of oleandrin (in vitro assay according to Example 26) versus oclacitinib (experimental data herein) upon particular cytokines using DN82 and canine fibroblasts.

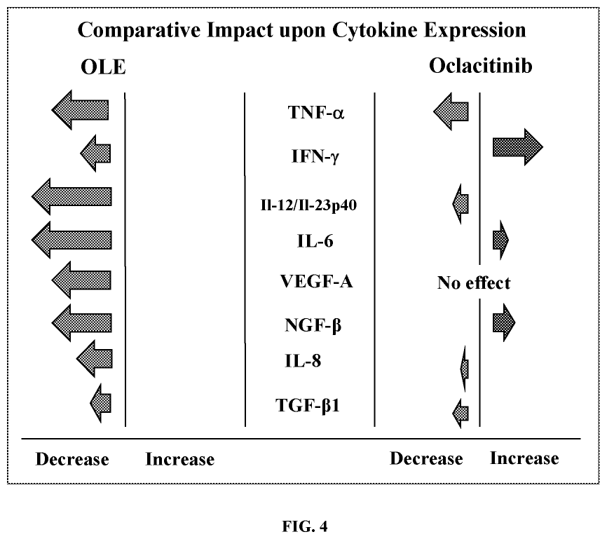

### DETAILED DESCRIPTION OF THE INVENTION

The invention provides a method of treating pruritus in an animal or human by chronic or acute administration of one or more effective doses of oleandrin-containing composition (OCC). The composition is administered according to a dosing regimen best suited for the subject, the suitability of the dose and dosing regimen to be determined clinically according to conventional clinical practices and clinical treatment endpoints for pruritus.

As used herein, the term “subject” is taken to mean humans and warm blooded animals such as mammals, for example, dog, cat, cow, horse, sheep, goat, pig, llama, alpaca, buffalo, deer, elk, giraffe, camel, or other hair-bearing or fur-bearing animal susceptible to suffering from pruritus.

A subject treated according to the invention will exhibit a therapeutic (clinically beneficial) response. By “therapeutic response” is meant that a subject having pruritus will a) scratch or lick the itchy skin less often; and/or b) will stop scratching or licking a patch of skin that was itchy prior to treatment. The therapeutic response can be a full or partial therapeutic response.

A dosing regimen includes a therapeutically relevant dose (or effective dose) of OCC administered according to a dosing schedule. A therapeutically relevant dose, therefore, is a therapeutic dose at which a therapeutic response of pruritus to treatment with OCC is observed and at which a subject can be administered the OCC without an excessive amount of unwanted or deleterious side effects. A therapeutically relevant dose is non-lethal to a subject, even though it may cause some side effects. It is a dose at which the level of clinical benefit to the subject being administered the OCC exceeds the level of deleterious side effects experienced by the subject due to administration of the OCC or component(s) thereof.

A therapeutically relevant dose will vary from subject to subject according to a variety of established pharmacologic, pharmacodynamic and pharmacokinetic principles. A therapeutically relevant daily dose will typically be in the range of about 0.1 micrograms to 100 micrograms, about 0.1 microg to about 500 microg, about 0.1 to about 100 microg per kg of bodyweight, about 15 to about 25 microg/kg, about 25 to about 50 microg/kg, about 50 to about 100 microg/kg, about 100 to about 200 microg/kg, about 200 to about 500 microg/kg, about 10 to about 750 microg/kg, about 16 to about 640 microg/kg, about 15 to about 750 microg/kg, about 15 to about 700 microg/kg, about 15 to about 650 microg/kg, about 0.05 to about 10 microg/Kg, about 0.1 to about 7.5 microg/Kg, about 0.1 to about 5 microg/Kg, or about 0.5 to about 5 microg/Kg bodyweight. Such dose can be administered one to five times daily.

Oleandrin may be administered to ruminant animals including ruminants include cattle, sheep, goats, buffalo, deer, elk, giraffes, and camels. For ruminant animals, the young animals have a different digestive tract than adult animals. Accordingly, the dose of oleandrin (microg of oleandrin per Kg of bodyweight) may be different in a young animal as compared to an adult animal of the same species. For example, a calf may require a different dose than a cow in order to benefit from oleandrin therapy. A veterinary clinician will be able to use known dose escalation or de-escalation protocols to determine a safe and effective dose to be administered.

A therapeutically relevant dose can be administered according to any dosing regimen typically used in the treatment of pruritus. A therapeutically relevant dose can be administered once, twice, thrice, or more, or continuously daily. It can be administered every other day, every third day, every fourth day, every fifth day, semiweekly, weekly, biweekly, every three weeks, every four weeks, monthly, bimonthly, semimonthly, every three months, every four months, semiannually, annually, or according to a combination of any of the above to arrive at a suitable dosing schedule. For example, a therapeutically relevant dose can be administered one or more times daily (up to 10 times daily for the highest dose) for one or more weeks.

Oleandrin may be included in feed and/or liquid administered to an animal. Oleandrin may be included in any feed format including solid feed, liquid feed, or gel feed. The solid feed may be loose granules, pellets, foodstuff, salt block, or other such feed used to feed animals.

The solid feed may comprise oleandrin and at least one feedstuff. Suitable feedstuffs include Whole cottonseed, cottonseed hulls, cottonseed meal, soybean meal, soybean hulls, corn gluten feed, hominy feed, dried distiller's grains, and rice mill feed are examples of commodity feedstuffs. Additional ingredients that may be included are selected from the group consisting of silage, nutritious supplement, vitamin, mineral, salt, grain (wheat, barley, oat, corn), fiber, hay, alfalfa, rye grass, beet, molasses, blood meal, bone meal, yeast, brome grass, canary grass, tomato, carrot, peas, pea vine hay, safflower, sage brush, sorghum, cheatgrass, clover, fat, grape, hominy, hops, meadow hay, sundan grass, sunflower, timothy hay, meat meal, milo, orange, orchard grass, potato, navy beans, peanut, prairie hay, rape meal, soybean, protein, meat, animal protein, plant protein, and combinations of any thereof.

The liquid feed comprises oleandrin, at least one liquid, and at least one nutrient. The liquid can be water, fermentation broth, milk, or milk substitute or other such liquid suitable for administration to an animal.

Given the bitter taste of oleandrin and oleander extracts, an oral, buccal, sublingual, or orodispersible composition administered to an animal can include one or more taste-masking agents. A sweetener, e.g. molasses, is advantageously included in a feed.

Oleandrin can also be included in water or other liquid given to the subject.

A composition can also include one or more additives suitable for administration to animals. For example, ammonium sulfate, calcium carbonate, sodium chloride, defluorinated phosphate, diammonium phosphate, dicalcium phosphate, limestone, monoammonium phosphate, monocalcium phosphate, sodium tripolyphos, urea, or any combination thereof may be used as additive.

Proof of the antipruritic activity of oleandrin was established in vitro according to Example 13 by measuring its effect upon on the expression of key cytokines, chemokines, and other factors that are part of the innate immune system. An in vitro model of human peripheral blood mononuclear cells was used to determine effects the effects of OCC under different culture conditions: normal (no inflammation) and inflamed by lipopolysaccharide (LPS).

T_{H}17 cells are pathogenically involved in inflammatory diseases. Il-25 aids in the control of adaptive immune responses in allergic diseases. IL-22, IL-23, and IL-31 are involved in the JAK (Janus Kinase)-STAT pathway. IL-33 is associated with allergic or psoriatic inflammation. IL-10 is a master regulator of host-response and resistance to pathogens. IFN-γ regulates immunity and bridges innate and specific immune responses. MIP-10 recruits early response inflammatory cells, promotes wound healing, inhibits stem cells, and maintains effector responses. IL-1ra induces anti-inflammatory response to minimize tissue damage. G-CSF Promote white blood cell production in the bone marrow. IL-6 stimulates acute phase immune response and hematopoiesis.

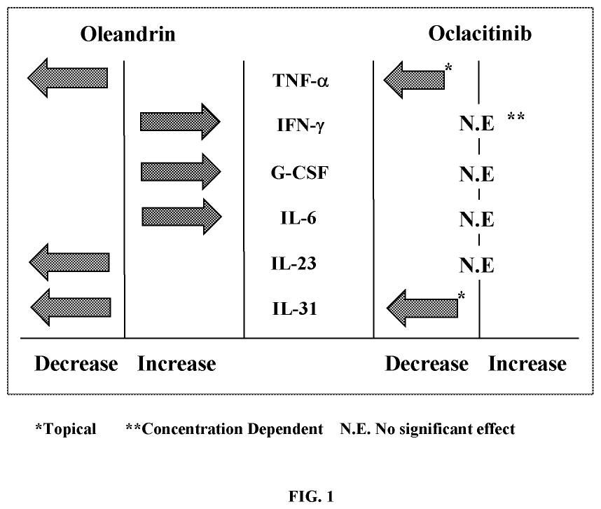

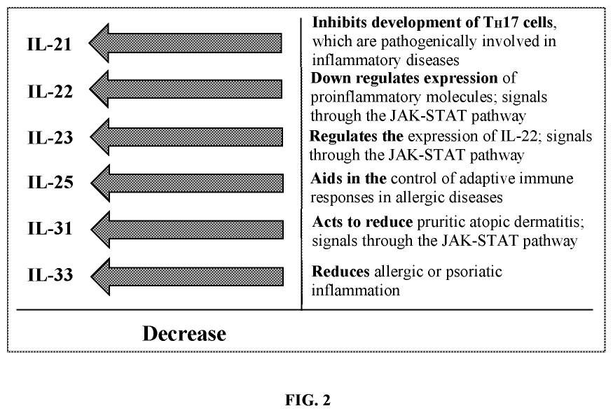

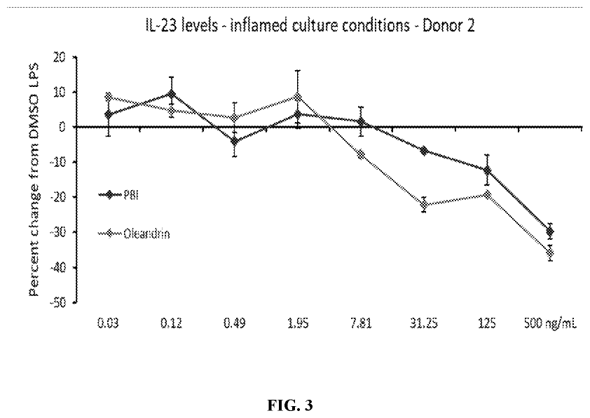

Cells were evaluated for immune activation marks CD69, CD25, and CD107a, and culture supernatants were tested for cytokines. It was determined that oleandrin (evaluated in pure form and as part of a supercritical fluid extract (PBI-05204) of Nerium oleander) suppressed the formation/expression of IL-21, IL-22, IL-23, IL-25, IL-31, IL-33, and TNF-α and induced expression of IFN-γ, G-CSF, and IL-6. Other chemokines and cytokines were also affected. Both PBI-05204 and oleandrin directly activated Natural Killer (NK) cells and monocytes and triggered increased production of cytokines. Under inflammatory conditions, many cytokines were controlled at similar levels as in cultures treated with PBI and oleandrin without inflammation. PBI-05204 triggered higher levels of some cytokines than oleandrin. Both products increased T cell cytotoxic attack on malignant target cells, strongest by PBI-05204. The results show that PBI-05204 and oleandrin directly activate innate immune cells, enhance anti-viral immune responses through NK cell activation and IFN-γ levels, and modulate immune responses under inflamed conditions. Results are depicted in FIGS. 1-3.

A comparison of the activity of oleandrin and oleander extract versus of oclacitinib was conducted in DN82 and canine fibroblasts according to Example. The objective of this study was to perform testing for cytokines involved in dermal inflammatory reactions and to document and compare the effects of an oleander extract (OE), oleandrin, and oclacitinib on biomarkers relevant for allergic reactions. The effects of these compounds under inflamed culture conditions are of direct importance to treatment of canine atopic dermatitis. Testing involved two canine cell types important for initiating, regulating, and resolving dermal allergic reactions: Primary canine dermal fibroblasts and the canine DH82 macrophage cell line. Under inflamed conditions, OE and oleandrin downregulated key cytokines secreted by canine dermal fibroblasts and the DH82 macrophage cell line; all of which are treatment targets in dermatitis. In the DH82 macrophage cultures, the most noteworthy reductions involved IL-6, IL-12/IL-23p40, IFN-γ; TNF-α, VEGF, and NGF-β. Oclacitinib triggered reductions of some cytokines involved in allergic reactions, including TGF-β1, IL12/IL-23p40, and TNF-α; however, these reductions were less robust than the reductions triggered by OE and oleandrin, and accompanied by increases in other cytokines involved in dermal inflammation, including IL-6, IFN-γ, and NGF-β. In cultures of primary dermal fibroblasts, OE and oleandrin reduced the levels of IL-8 and MCP-1, whereas oclacitinib had little or no effect. It was determined that OE and oleandrin directly modulate immune responses under inflamed conditions. Moreover, OE and oleandrin appear to provide a more beneficial overall cytokine regulation than oclacitinib under inflamed culture conditions. The overall pattern of reduced cytokine production under inflamed conditions was favorable for OE and oleandrin. Oclacitinib, under both unstressed and inflamed culture conditions, induced cytokines that are thought to be undesirable in dermal allergies. Both OE and oleandrin directly activated Natural Killer (NK) cells and monocytes and triggered increased production of cytokines.

Proof of the in vivo antipruritic efficacy of oleandrin (oleandrin-containing composition) was established according to Examples 5, 12, 14, 15, 24, and 28. Subjects presenting with pruritus were administered OCC on a daily basis. The extent of relief from itching was determined as evidenced by reduced scratching, reduced redness, and/or reduced inflammation of an itchy patch of skin. For non-systemic (topical) administration, complete or substantial relief from itching occurred almost immediately or in less than about ten minutes. For systemic administration (oral, buccal, sublingual), relief from itching occurred within about 10 min to 3 hours. Continued administration of OCC on a daily basis after resolution of the initial case of pruritus prevented further occurrence of pruritus.

In particular, safe and efficacious oral administration to dogs was established according to Example 28, wherein oleandrin (as included in an oleander extract) was administered orally to dogs as either part of their meal or in between meals. The product was administered to dogs suffering from pruritus or atopic dermatitis 1-2 times daily at doses ranging from 2-6 or 3-5 microg oleandrin/Kg of bodyweight. The dogs all exhibited significant reduction in itching and scratching, the improvements became evident in 1-3 days, and they were maintained throughout the dosing period.

Daily administration is preferred. For breakthrough episodes of AD or pruritus, the OCC is preferably administered topically. For maintenance doses to control, reduce or eliminate AD or pruritus on an ongoing basis, the OCC is preferably administered orally, topically, or via a combination of both routes. Oral administration between meals instead of as part of a meal is preferred. For initial treatment, the product can be administered topically or topically and orally to bring the AD or pruritus under control. For a continuous treatment to keep the AD or pruritus under control, the product is preferably administered orally, and any breakthrough episodes can be further treated by topical administration in combination with oral administration.

Accordingly, the invention provides a method of treating pruritus in a subject, the method comprising administering one or more effective doses of oleandrin (OCC) to a subject having pruritus. The invention also provides a method of preventing recurrence of pruritus, the method comprising administering one or more effective doses of OCC to a subject having already experienced resolution of a prior occurrence of pruritus, thereby preventing recurrence of pruritus in the subject. The administration can be according to any of the dosing regimens described herein.

The concentrations of oleandrin evaluated in the in vitro assays are clinically relevant in terms of dosing and plasma concentration.

Proof of the safety of the oleandrin-containing composition was provided by in vitro cellular assays for determining the release of lactate dehydrogenase after exposure of said cells to solutions containing different concentrations of oleandrin. It was determined that up to concentrations of 1 microg/mL, there was no additional toxicity over control vehicle.

In some embodiments, an OCC comprises a) oleandrin, or b) oleandrin and one or more other compounds extracted from Nerium species. The oleandrin may be present as part of an extract of Nerium species, which extract may be a a) supercritical fluid extract; b) hot-water extract; c) organic solvent extract; d) aqueous organic solvent extract; e) extract using supercritical fluid, optionally plus at least one organic solvent (extraction modifier); f) extract using subcritical liquid, optionally plus at least one organic solvent (extraction modifier); or g) any combination of any two or more of said extracts.

PBI-05204 (as described herein and in U.S. Pat. No. 8,187,644 B2 to Addington, which issued May 29, 2012, U.S. Pat. No. 7,402,325 B2 to Addington, which issued Jul. 22, 2008, U.S. Pat. No. 8,394,434 B2 to Addington et al, which issued Mar. 12, 2013, the entire disclosures of which are hereby incorporated by reference) comprises cardiac glycoside (oleandrin, OL) and triterpenes (oleanolic acid (OA), ursolic acid (UA) and betulinic acid (BA)) as the primary pharmacologically active components. The molar ratio of OL to total triterpene is about 1:(10-96). The molar ratio of OA:UA:BA is about 7.8:7.4:1, about 3:2.2:1, about 2-8 (OA):about 2-8 (UA):about 0.1-1 (BA):about 0.5-1.5 (OL); or about 3-6 (OA):about 3-6 (UA):about 0.3-8 (BA):about 0.7-1.2 (OL); or about 4-5 (OA):about 4-5 (UA):about 0.4-0.7 (BA):about 0.9-1.1 (OL); about 4.6 (OA):about 4.4 (UA):about 0.6 (BA):about 1 (OL); about 9-12: up to about 2: up to about 2, or about 10:about 1:about 1, or about 9-12:about 0.1-2:about 0.1-2, or about 9-11:about 0.5-1.5:about 0.5-1.5, or about 9.5-10.5:about 0.75-1.25:about 0.75-1.25, or about 9.5-10.5:about 0.8-1.2:about 0.8-1.2, or about 9.75-10.5:about 0.9-1.1:about 0.9-1.1.

OCCs comprising oleandrin as the sole antipruritic agent are within the scope of the invention. OCCs comprising oleandrin and plural triterpenes as the antipruritic agents are within the scope of the invention. In some embodiments, the OCC comprises oleandrin, oleanolic acid (free acid, salt, derivative or prodrug thereof), ursolic acid (free acid, salt, derivative or prodrug thereof), and betulinic acid (free acid, salt, derivative or prodrug thereof). The molar ratios of the compounds are as described herein.

The OCCS of the invention can be administered as primary anti-pruritus therapy, adjunct anti-pruritus therapy, or co-anti-pruritus therapy. Methods of the invention include separate administration or coadministration of the OCC with at least one other known anti-pruritus composition, meaning the OCC of the invention can be administered before, during or after administration of a known anti-pruritus composition (compound(s)) or of a composition for treating symptoms associated with pruritus.

The one or more other therapeutic agents can be administered at doses and according to dosing regimens that are clinician-recognized as being therapeutically effective or at doses that are clinician-recognized as being sub-therapeutically effective. The clinical benefit and/or therapeutic effect provided by administration of a combination of OCC and one or more other therapeutic compositions can be additive or synergistic, such level of benefit or effect being determined by comparison of administration of the combination to administration of the individual antipruritic composition component(s) and one or more other therapeutic agents. The one or more other therapeutic agents can be administered at doses and according to dosing regimens as suggested or described by the Food and Drug Administration (Center for Veterinary Medicine), World Health Organization, European Medicines Agency (Veterinary Medicines Division), Australian Pesticides and Veterinary Medicines Authority (APVMA), Pan American Health Organization (Veterinary Public Health Program), Agricultural Compounds and Veterinary Medicines Authority (New Zealand) or the various Ministries of Health worldwide.

A composition containing oclacitinib (present as free base or salt, in particular maleate salt) is made by mixing oleandrin, oclacitinib, and excipients to form the composition. Where a dose of the composition is intended to be administered in a single dosage form, the composition will contain amounts of the components sufficient to provide a unit dose of about 0.05-100 microg (or about 0.1-50 microg, about 0.1-25 microg, 0.1-15 microg, 0.1-10 microg, or 0.1-5 microg) of oleandrin and about 0.5-50 mg (or about 1-50 mg, about 1-40 mg, about 1.5-38 mg, about 1.8-32 mg) of oclacitinib (free base equivalents). Such composition can be administered one to four times daily or two to four times daily, once daily, or twice daily. The unit oral dose of oclacitinib can be further adjusted so that it is administered twice daily for about 1-8 weeks or about 2-6 weeks for treatment of pruritus, and then administered once daily for prevention of recurrence of pruritus. Daily oral doses of oclacitinib of about 0.6 mg/kg bodyweight, about 1.8 mg/kg bodyweight, or about 3.0 mg/kg bodyweight are suitable.

One or more compound(s) present in the pharmaceutical composition can be present in their unmodified form, salt form, derivative form or a combination thereof. As used herein, the term “derivative” is taken to mean: a) a chemical substance that is related structurally to a first chemical substance and theoretically derivable from it; b) a compound that is formed from a similar first compound or a compound that can be imagined to arise from another first compound, if one atom of the first compound is replaced with another atom or group of atoms; c) a compound derived or obtained from a parent compound and containing essential elements of the parent compound; or d) a chemical compound that may be produced from first compound of similar structure in one or more steps. For example, a derivative may include a deuterated form, oxidized form, dehydrated, unsaturated, polymer conjugated or glycosylated form thereof or may include an ester, amide, lactone, homolog, ether, thioether, cyano, amino, alkylamino, sulfhydryl, heterocyclic, heterocyclic ring-fused, polymerized, pegylated, benzylidenyl, triazolyl, piperazinyl or deuterated form thereof.

As used herein, the term “oleandrin” is taken to mean all known forms of oleandrin unless otherwise specified. Oleandrin can be present in racemic, optically pure or optically enriched form. Nerium sp. plant material can be obtained, for example, from commercial plant suppliers such as Aldridge Nursery, Atascosa, Texas.

The supercritical fluid (SCF) extract can be prepared as detailed in U.S. Pat. Nos. 7,402,325, 8,394,434, 8,187,644, or PCT International Publication No. WP 2007/016176 A2, the entire disclosures of which are hereby incorporated by reference. Extraction can be conducted with supercritical carbon dioxide in the presence or absence of a modifier (organic solvent) such as ethanol.

A hot-water extract is available under the tradename ANVIRZEL™ (Nerium Biotechnology, Inc., San Antonio, TX; Salud Integral Medical Clinic, Tegucigalpa, Honduras; www.saludintegral.com; www.anvirzel.com) as a liquid dosage form. ANVIRZEL™ comprises oleandrin, oleandrigenin, polysaccharides extracted (hot water extraction) from Nerium oleander. Commercially available vials comprise about 150 mg of oleander extract as a freeze-dried powder (prior to reconstitution with water before administration) which comprises about 200 to about 900 microg of oleandrin, about 500 to about 700 microg of oleandrigenin, and polysaccharides extracted from Nerium oleander. Said vials may also include pharmaceutical excipients such as at least one osmotic agent, e.g. mannitol, sodium chloride, at least one buffering agent, e.g. sodium ascorbate with ascorbic acid, at least one preservative, e.g. propylparaben, methylparaben.

Other extracts containing cardiac glycoside, especially oleandrin, can be prepared by various different processes. An extract can be prepared according to the process developed by Dr. Huseyin Ziya Ozel (U.S. Pat. No. 5,135,745) describes a procedure for the preparation of a hot water extract. The aqueous extract reportedly contains several polysaccharides with molecular weights varying from 2 KD to 30 KD, oleandrin, oleandrigenin, odoroside and neritaloside. The polysaccharides reportedly include acidic homopolygalacturonans or arabinogalaturonans. U.S. Pregrant Patent Application Publication No. 20040247660 to Singh et al. discloses the preparation of a protein stabilized liposomal formulation of oleandrin for use in the treatment of cancer. U.S. Pregrant Patent Application Publication No. 20050026849 to Singh et al. discloses a water soluble formulation of oleandrin containing a cyclodextrin. U.S. Pregrant Patent Application Publication No. 20040082521 to Singh et al. discloses the preparation of protein stabilized nanoparticle formulations of oleandrin from the hot-water extract.

The extracts also differ in their polysaccharide and carbohydrate content. The hot water extract contains 407.3 glucose equivalent units of carbohydrate relative to a standard curve prepared with glucose while analysis of the SCF CO₂ extract found carbohydrate levels that were found in very low levels that were below the limit of quantitation. The amount of carbohydrate in the hot water extract of Nerium oleander was, however, at least 100-fold greater than that in the SCF CO₂ extract. The polysaccharide content of the SCF extract can be 0%, <0.5%, <0.1%, <0.05%, or <0.01% wt. In some embodiments, the SCF extract excludes polysaccharide obtained during extraction of the plant mass.

| Nerium oleander | Polysaccharide content (μg glucose |
|---|---|
| preparation | equivalents/mg of plant extract) |
| Hot water extract | 407.3 ± 6.3 |
| SCF CO₂ extract | BLQ (below limit of quantitation) |

The partial compositions of the SCF CO₂ extract and hot water extract were determined by DART TOF-MS (Direct Analysis in Real Time Time of Flight Mass Spectrometry) on a JEOL AccuTOF-DART mass spectrometer (JEOL USA, Peabody, MA, USA).

The SCF extract of Nerium species or Thevetia species is a mixture of pharmacologically active compounds, such as oleandrin and triterpenes. The extract obtained by the SCF process is a substantially water-insoluble, viscous semi-solid (after solvent is removed) at ambient temperature. The SCF extract comprises many different components possessing a variety of different ranges of water solubility. The extract from a supercritical fluid process contains by weight a theoretical range of 0.9% to 2.5% wt of oleandrin or 1.7% to 2.1% wt of oleandrin or 1.7% to 2.0% wt of oleandrin. SCF extracts comprising varying amount of oleandrin have been obtained. In one embodiment, the SCF extract comprises about 2% by wt. of oleandrin. The SCF extract contains a 3-10 fold higher concentration of oleandrin than the hot-water extract. This was confirmed by both HPLC as well as LC/MS/MS (tandem mass spectrometry) analyses.

The SCF extract comprises oleandrin and the triterpenes oleanolic acid, betulinic acid and ursolic acid and optionally other components as described herein. The content of oleandrin and the triterpenes can vary from batch to batch; however, the degree of variation is not excessive. For example, a batch of SCF extract (PBI-05204) was analyzed for these four components and found to contain the following approximate amounts of each.

| | | Oleanolic | Ursolic | Betulinic |
|---|---|---|---|---|
| | Oleandrin | acid | acid | acid |
| Content of component | 20 | 73 | 69 | 9.4 |
| (mg/g of SCF extract) | | | | |
| Content of component | 2 | 7.3 | 6.9 | 0.94 |
| (% wt WRT g of SCT | | | | |
| extract) | | | | |
| Content of component | 34.7 | 160 | 152 | 20.6 |
| (mmole/g of SCF | | | | |
| extract) | | | | |
| Molar ratio of | 1 | 4.6 | 4.4 | 0.6 |
| component WRT | | | | |
| oleandrin | | | | |

WRT denotes “with respect to”.

The content of the individual components may vary by ±25%, ±20%, ±15%, ±10% or ±5% relative to the values indicated. Accordingly, the content of oleandrin in the SCF extract would be in the range of 20 mg±5 mg (which is ±25% of 20 mg) per mg of SCF extract.

Oleandrin, oleanolic acid, ursolic acid, betulinic acid and derivatives thereof can also be purchased from Sigma-Aldrich (www.sigmaaldrich.com; St. Louis, MO, USA). Digoxin is commercially available from HIKMA Pharmaceuticals International LTD (NDA N012648, elixir, 0.05 mg/mL; tablet, 0.125 mg, 0.25 mg), VistaPharm Inc. (NDA A213000, elixir, 0.05 mg/mL), Sandoz Inc. (NDA A040481, injectable, 0.25 mg/mL), West-Ward Pharmaceuticals International LTD (NDA A083391, injectable, 0.25 mg/mL), Covis Pharma BV (NDA N009330, 0.1 mg/mL, 0.25 mg/mL), Impax Laboratories (NDA A078556, tablet, 0.125 mg, 0.25 mg), Jerome Stevens Pharmaceuticals Inc. (NDA A076268, tablet, 0.125 mg, 0.25 mg), Mylan Pharmaceuticals Inc. (NDA A040282, tablet, 0.125 mg, 0.25 mg), Sun Pharmaceutical Industries Inc. (NDA A076363, tablet, 0.125 mg, 0.25 mg), Concordia Pharmaceuticals Inc. (NDA A020405, tablet, 0.0625, 0.125 mg, 0.1875 mg, 0.25 mg, 0.375 mg, 0.5 mg, LANOXIN), GlaxoSmithKline LLC (NDA 018118, capsule, 0.05 mg, 0.1 mg, 0.15 mg, 0.2 mg, LANOXICAPS).

As used herein, the individually named triterpenes can independently be selected upon each occurrence in their native (unmodified, free acid) form, in their salt form, in derivative form, prodrug form, or a combination thereof. Compositions containing and methods employing deuterated forms of the triterpenes are also within the scope of the invention.

The composition can be formulated in any suitable pharmaceutically acceptable dosage form. Parenteral, otic, ophthalmic, nasal, inhalable, buccal, sublingual, enteral, topical, oral, peroral, transdermal, and injectable dosage forms are particularly useful. Particular dosage forms include a solid or liquid dosage forms. Exemplary suitable dosage forms include tablet, capsule, pill, caplet, troche, sache, solution, suspension, dispersion, vial, bag, bottle, injectable liquid, i.v. (intravenous), i.m. (intramuscular) or i.p. (intraperitoneal) administrable liquid and other such dosage forms known to the artisan of ordinary skill in the pharmaceutical sciences.

Suitable dosage forms for administering oleandrin (or digoxin) to an animal can be made according to known procedures wherein oleandrin (or digoxin) is used in place of another drug: Klink et al. (“Formulations of Veterinary Dosage Forms” in Development and Formulation of Veterinary Dosage Forms, 2^{nd} ed., Eds. G. E. Hardee and J. D. Baggot, New York, CRC Press, 1998), Foster et al. (“Veterinary Dosage Forms” in Encyclopedia of Pharmaceutical Science and Technology, 4^{th} ed., Eds. J. Swarbrick, New York, CRC Press, 2015).

The desired dose for oral administration is up to 5 dosage forms although as few as one and as many as ten dosage forms may be administered as a single dose. Doses will be administered according to dosing regimens that may be predetermined and/or tailored to achieve specific therapeutic response or clinical benefit in an animal.

The cardiac glycoside can be present in a dosage form in an amount sufficient to provide an animal with an initial dose of oleandrin of about 20 to about 100 microg, about 12 microg to about 300 microg, or about 12 microg to about 120 microg. For example, a dosage form can comprise about 20 of oleandrin to about 100 microg, about 0.01 microg to about 100 mg or about 0.01 microg to about 100 microg oleandrin, oleandrin extract or extract of Nerium sp. containing oleandrin.

The composition can be included in an oral dosage form. Some embodiments of the dosage form are not enteric coated and release their charge of cardiac glycoside composition within a period of 0.5 to 1 hours or less. Some embodiments of the dosage form are enteric coated and release their charge of cardiac glycoside downstream of the stomach, such as from the jejunum, ileum, small intestine, and/or large intestine (colon). Enterically coated dosage forms will release cardiac glycoside into the systemic circulation within 1-10 hr after oral administration.

The composition can be included in a rapid release, immediate release, controlled release, sustained release, prolonged release, extended release, burst release, continuous release, slow release, or pulsed release dosage form or in a dosage form that exhibits two or more of those types of release. The release profile of cardiac glycoside from the dosage form can be a zero order, pseudo-zero, first order, pseudo-first order or sigmoidal release profile. The plasma concentration profile for cardiac glycoside in an animal to which the CGCC is administered can exhibit one or more maxima.

The anticipated oleandrin plasma concentration (Cmax or Cavg as measure in a 24-h period) will be in the range of about 0.005 to about 5 ng/ml, about 0.005 to about 4 ng/mL, about 0.005 to about 3 ng/mL, about 0.005 to about 2 ng/mL, or about 0.005 to about 2 ng/mL. A veterinary clinician will use known dose escalation and de-escalation protocols to determine the appropriate dose of oleandrin or digoxin to be safely administered per day.

It should be noted that a compound herein might possess one or more functions in a composition or formulation of the invention. For example, a compound might serve as both a surfactant and a water miscible solvent or as both a surfactant and a water immiscible solvent.

A liquid composition can comprise one or more pharmaceutically acceptable liquid carriers. The liquid carrier can be an aqueous, non-aqueous, polar, non-polar, and/or organic carrier. Liquid carriers include, by way of example and without limitation, a water miscible solvent, water immiscible solvent, water, buffer and mixtures thereof.

As used herein, the terms “water soluble solvent” or “water miscible solvent”, which terms are used interchangeably, refer to an organic liquid which does not form a biphasic mixture with water or is sufficiently soluble in water to provide an aqueous solvent mixture containing at least five percent of solvent without separation of liquid phases. The solvent is suitable for administration to animals. Exemplary water soluble solvents include, by way of example and without limitation, PEG (poly(ethylene glycol)), PEG 400 (poly(ethylene glycol having an approximate molecular weight of about 400), ethanol, acetone, alkanol, alcohol, ether, propylene glycol, glycerin, triacetin, poly(propylene glycol), PVP (poly(vinyl pyrrolidone)), dimethylsulfoxide, N,N-dimethylformamide, formamide, N,N-dimethylacetamide, pyridine, propanol, N-methylacetamide, butanol, soluphor (2-pyrrolidone), pharmasolve (N-methyl-2-pyrrolidone).

As used herein, the terms “water insoluble solvent” or “water immiscible solvent”, which terms are used interchangeably, refer to an organic liquid which forms a biphasic mixture with water or provides a phase separation when the concentration of solvent in water exceeds five percent. The solvent is suitable for administration to animals. Exemplary water insoluble solvents include, by way of example and without limitation, medium/long chain triglycerides, oil, castor oil, corn oil, vitamin E, vitamin E derivative, oleic acid, fatty acid, olive oil, softisan 645 (Diglyceryl Caprylate/Caprate/Stearate/Hydroxy stearate adipate), miglyol, captex (Captex 350: Glyceryl Tricaprylate/Caprate/Laurate triglyceride; Captex 355: Glyceryl Tricaprylate/Caprate triglyceride; Captex 355 EP/NF: Glyceryl Tricaprylate/Caprate medium chain triglyceride).

Suitable solvents are listed in the “International Conference on Harmonisation of Technical Requirements for Registration of Pharmaceuticals for Human Use (ICH) guidance for industry Q3C Impurities: Residual Solvents” (1997), which makes recommendations as to what amounts of residual solvents are considered safe in pharmaceuticals. Exemplary solvents are listed as class 2 or class 3 solvents. Class 3 solvents include, for example, acetic acid, acetone, anisole, 1-butanol, 2-butanol, butyl acetate, tert-butlymethyl ether, cumene, ethanol, ethyl ether, ethyl acetate, ethyl formate, formic acid, heptane, isobutyl acetate, isopropyl acetate, methyl acetate, methyl-1-butanol, methylethyl ketone, methylisobutyl ketone, 2-methyl-1-propanol, pentane, 1-pentanol, 1-propanol, 2-propanol, or propyl acetate.

Other materials that can be used as water immiscible solvents in the invention include: Captex 100: Propylene Glycol Dicaprate; Captex 200: Propylene Glycol Dicaprylate/Dicaprate; Captex 200 P: Propylene Glycol Dicaprylate/Dicaprate; Propylene Glycol Dicaprylocaprate; Captex 300: Glyceryl Tricaprylate/Caprate; Captex 300 EP/NF: Glyceryl Tricaprylate/Caprate Medium Chain Triglycerides; Captex 350: Glyceryl Tricaprylate/Caprate/Laurate; Captex 355: Glyceryl Tricaprylate/Caprate; Captex 355 EP/NF: Glyceryl Tricaprylate/Caprate Medium Chain Triglycerides; Captex 500: Triacetin; Captex 500 P: Triacetin (Pharmaceutical Grade); Captex 800: Propylene Glycol Di (2-Ethythexanoate); Captex 810 D: Glyceryl Tricaprylate/Caprate/Linoleate; Captex 1000: Glyceryl Tricaprate; Captex CA: Medium Chain Triglycerides; Captex MCT-170: Medium Chain Triglycerides; Capmul GMO: Glyceryl Monooleate; Capmul GMO-50 EP/NF: Glyceryl Monooleate; Capmul MCM: Medium Chain Mono- & Diglycerides; Capmul MCM C8: Glyceryl Monocaprylate; Capmul MCM C10: Glyceryl Monocaprate; Capmul PG-8: Propylene Glycol Monocaprylate; Capmul PG-12: Propylene Glycol Monolaurate; Caprol 10G10O: Decaglycerol Decaoleate; Caprol 3GO: Triglycerol Monooleate; Caprol ET: Polyglycerol Ester of Mixed Fatty Acids; Caprol MPGO: Hexaglycerol Dioleate; Caprol PGE 860: Decaglycerol Mono-, Dioleate.

As used herein, a “surfactant” refers to a compound that comprises polar or charged hydrophilic moieties as well as non-polar hydrophobic (lipophilic) moieties; i.e., a surfactant is amphiphilic. The term surfactant may refer to one or a mixture of compounds. A surfactant can be a solubilizing agent, an emulsifying agent or a dispersing agent. A surfactant can be hydrophilic or hydrophobic.

The hydrophilic surfactant can be any hydrophilic surfactant suitable for use in pharmaceutical compositions. Such surfactants can be anionic, cationic, zwitterionic or non-ionic, although non-ionic hydrophilic surfactants are presently preferred. As discussed above, these non-ionic hydrophilic surfactants will generally have HLB values greater than about 10. Mixtures of hydrophilic surfactants are also within the scope of the invention.

Similarly, the hydrophobic surfactant can be any hydrophobic surfactant suitable for use in pharmaceutical compositions. In general, suitable hydrophobic surfactants will have an HLB value less than about 10. Mixtures of hydrophobic surfactants are also within the scope of the invention.

Examples of additional suitable solubilizer include: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide); amides, such as 2-pyrrolidone, 2-piperidone, caprolactam, N-alkylpyrrolidone, N-hydroxyalkylpyrrolidone, N-alkylpiperidone, N-alkylcaprolactam, dimethylacetamide, and polyvinypyrrolidone; esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, caprolactone and isomers thereof, valerolactone and isomers thereof, butyrolactone and isomers thereof, and other solubilizers known in the art, such as dimethyl acetamide, dimethyl isosorbide (Arlasolve DMI (ICI)), N-methyl pyrrolidones (Pharmasolve (ISP)), monooctanoin, diethylene glycol nonoethyl ether (available from Gattefosse under the trade name Transcutol), and water. Mixtures of solubilizers are also within the scope of the invention.

Except as indicated, compounds mentioned herein are readily available from standard commercial sources.

Although not necessary, the composition or formulation may further comprise one or more chelating agents, one or more preservatives, one or more antioxidants, one or more adsorbents, one or more acidifying agents, one or more alkalizing agents, one or more antifoaming agents, one or more buffering agents, one or more colorants, one or more electrolytes, one or more salts, one or more stabilizers, one or more tonicity modifiers, one or more diluents, or a combination thereof.

The composition of the invention can also include oils such as fixed oils, peanut oil, sesame oil, cottonseed oil, corn oil and olive oil; fatty acids such as oleic acid, stearic acid and isostearic acid; and fatty acid esters such as ethyl oleate, isopropyl myristate, fatty acid glycerides and acetylated fatty acid glycerides. The composition can also include alcohol such as ethanol, isopropanol, hexadecyl alcohol, glycerol and propylene glycol; glycerol ketals such as 2,2-dimethyl-1,3-dioxolane-4-methanol; ethers such as poly(ethylene glycol) 450; petroleum hydrocarbons such as mineral oil and petrolatum; water; a pharmaceutically suitable surfactant, suspending agent or emulsifying agent; or mixtures thereof.

One or more of the components of the formulation can be present in its free base, free acid or pharmaceutically or analytically acceptable salt form. As used herein, “pharmaceutically or analytically acceptable salt” refers to a compound that has been modified by reacting it with an acid as needed to form an ionically bound pair. Examples of acceptable salts include conventional non-toxic salts formed, for example, from non-toxic inorganic or organic acids. Suitable non-toxic salts include those derived from inorganic acids such as hydrochloric, hydrobromic, sulfuric, sulfonic, sulfamic, phosphoric, nitric and others known to those of ordinary skill in the art. The salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and others known to those of ordinary skill in the art. On the other hand, where the pharmacologically active ingredient possesses an acid functional group, a pharmaceutically acceptable base is added to form the pharmaceutically acceptable salt. Lists of other suitable salts are found in Remington's Pharmaceutical Sciences, 17^{th}. ed., Mack Publishing Company, Easton, PA, 1985, p. 1418, the relevant disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with tissues of animals and without excessive toxicity, irritation, allergic response, or any other problem or complication, commensurate with a reasonable benefit/risk ratio.

A dosage form can be made by any conventional means known in the pharmaceutical industry. A liquid dosage form can be prepared by providing at least one liquid carrier and CGCC composition in a container. One or more other excipients can be included in the liquid dosage form. A solid dosage form can be prepared by providing at least one solid carrier and CGCC composition. One or more other excipients can be included in the solid dosage form.

A dosage form can be packaged using conventional packaging equipment and materials. It can be included in a pack, bottle, via, bag, syringe, envelope, packet, blister pack, box, ampoule, or other such container.

The composition of the invention can be included in any dosage form. Particular dosage forms include solid or liquid dosage forms. Exemplary suitable dosage forms include tablet, capsule, pill, caplet, troche, sache, and other such dosage forms known to the artisan of ordinary skill in the pharmaceutical sciences.

The CGCC can further comprise at least one cardiac glycoside-metabolism inhibitor, at least one cardiac glycoside-digestion inhibitor, at least one enzyme inhibitor, or a combination thereof. A cardiac glycoside-metabolism inhibitor is a compound that inhibits metabolism of a cardiac glycoside. A cardiac glycoside-digestion inhibitor is a compound that inhibits digestion of a cardiac glycoside. An enzyme inhibitor is a compound that inhibits an enzyme. The metabolism or digestion can be caused by the animal or one or more microbes in the animal. These categories of inhibitors are herein referred to together more broadly as inhibitors. The purpose of said inhibitors is to reduce the rate of metabolism or digestion of the cardiac glycoside, thereby increasing the plasma concentration half-life of the cardiac glycoside in the animal.

In view of the above description and the examples below, one of ordinary skill in the art will be able to practice the invention as claimed without undue experimentation. The foregoing will be better understood with reference to the following examples that detail certain procedures for the preparation of embodiments of the present invention. All references made to these examples are for the purposes of illustration. The following examples should not be considered exhaustive, but merely illustrative of only a few of the many embodiments contemplated by the present invention.

### Example 1

### Supercritical Fluid Extraction of Powdered Oleander Leaves

### Method A. With Carbon Dioxide.

Powdered oleander leaves were prepared by harvesting, washing, and drying oleander leaf material, then passing the oleander leaf material through a comminuting and dehydrating apparatus such as those described in U.S. Pat. Nos. 5,236,132, 5,598,979, 6,517,015, and 6,715,705. The weight of the starting material used was 3.94 kg.

The starting material was combined with pure CO₂ at a pressure of 300 bar (30 MPa, 4351 psi) and a temperature of 50° C. (122° F.) in an extractor device. A total of 197 kg of CO₂ was used, to give a solvent to raw material ratio of 50:1. The mixture of CO₂ and raw material was then passed through a separator device, which changed the pressure and temperature of the mixture and separated the extract from the carbon dioxide.

The extract (65 g) was obtained as a brownish, sticky, viscous material having a nice fragrance. The color was likely caused by chlorophyll and other residual chromophoric compounds. For an exact yield determination, the tubes and separator were rinsed out with acetone and the acetone was evaporated to give an addition 9 g of extract. The total extract amount was 74 g. Based on the weight of the starting material, the yield of the extract was 1.88%. The content of oleandrin in the extract was calculated using high pressure liquid chromatography and mass spectrometry to be 560.1 mg, or a yield of 0.76%.

### Method B. With Mixture of Carbon Dioxide and Ethanol

Powdered oleander leaves were prepared by harvesting, washing, and drying oleander leaf material, then passing the oleander leaf material through a comminuting and dehydrating apparatus such as those described in U.S. Pat. Nos. 5,236,132, 5,598,979, 6,517,015, and 6,715,705. The weight of the starting material used was 3.85 kg.

The starting material was combined with pure CO₂ and 5% ethanol as a modifier at a pressure of 280 bar (28 MPa, 4061 psi) and a temperature of 50° C. (122° F.) in an extractor device. A total of 160 kg of CO₂ and 8 kg ethanol was used, to give a solvent to raw material ratio of 43.6 to 1. The mixture of CO₂, ethanol, and raw material was then passed through a separator device, which changed the pressure and temperature of the mixture and separated the extract from the carbon dioxide.

The extract (207 g) was obtained after the removal of ethanol as a dark green, sticky, viscous mass obviously containing some chlorophyll. Based on the weight of the starting material, the yield of the extract was 5.38%. The content of oleandrin in the extract was calculated using high pressure liquid chromatography and mass spectrometry to be 1.89 g, or a yield of 0.91%.

### Example 2

### Hot-Water Extraction of Powdered Oleander Leaves.

Hot water extraction is typically used to extract oleandrin and other active components from oleander leaves. Examples of hot water extraction processes can be found in U.S. Pat. Nos. 5,135,745 and 5,869,060.

A hot water extraction was carried out using 5 g of powdered oleander leaves. Ten volumes of boiling water (by weight of the oleander starting material) were added to the powdered oleander leaves and the mixture was stirred constantly for 6 hours. The mixture was then filtered and the leaf residue was collected and extracted again under the same conditions. The filtrates were combined and lyophilized. The appearance of the extract was brown. The dried extract material weighed about 1.44 g. 34.21 mg of the extract material was dissolved in water and subjected to oleandrin content analysis using high pressure liquid chromatography and mass spectrometry. The amount of oleandrin was determined to be 3.68 μg. The oleandrin yield, based on the amount of extract, was calculated to be 0.26%.

A suitable aqueous liquid composition was prepared by dissolving 300 mg of dried extract material (containing about 350-400 μg of oleandrin (OLE) per g of dried extract) in 20 mL of water to provide, thereby resulting in a concentration of 15 mg of extract/mL (about 17.5-20 μg OLE/mL). Solutions having higher and lower concentrations of OLE were made.

### Example 3

### Preparation of Veterinary Compositions.

### Method A. Cremophor-Based Drug Delivery System

The following ingredients were provided in the amounts indicated.

| | | Percent of Formulation |
|---|---|---|
| Reagent Name | Function | (% w/w) |
| OCC | Active agent | 3.7 |
| Vitamin E | Antioxidant | 0.1 |
| Labrasol | Surfactant | 9.2 |
| Ethanol | Co-solvent | 9.6 |
| Cremophor EL | Surfactant | 62.6 |
| Cremophor RH40 | Surfactant | 14.7 |

The excipients were dispensed into a jar and shook in a New Brunswick Scientific C24KC Refrigerated Incubator shaker for 24 hours at 60° C. to ensure homogeneity. The samples were then pulled and visually inspected for solubilization. Both the excipients and OCC were totally dissolved for all formulations after 24 hours.

### Method B. GMO/Cremophor-Based Drug Delivery System

The following ingredients were provided in the amounts indicated.

| | | Percent of Formulation |
|---|---|---|
| Reagent Name | Function | (% w/w) |
| OCC | Active agent | 4.7 |
| Vitamin E | Antioxidant | 0.1 |
| Labrasol | Surfactant | 8.5 |
| Ethanol | Co-solvent | 7.6 |
| Cremophor EL | Surfactant | 56.1 |
| Glycerol Monooleate | Surfactant | 23.2 |

The procedure of Method A was followed.

### Method C. Labrasol-Based Drug Delivery System

The following ingredients were provided in the amounts indicated.

| Reagent | | |
|---|---|---|
| Name | Function | Percent of Formulation (% w/w) |
| OCC | Active agent | 3.7 |
| Vitamin E | Antioxidant | 0.1 |
| Labrasol | Surfactant | 86.6 |
| Ethanol | Co-solvent | 9.6 |

The procedure of Method A was followed.

### Method D. Vitamin E-TPGS Based Micelle Forming System

The following ingredients were provided in the amounts indicated.

| Component | Function | Weight % (w/w) |
|---|---|---|
| Vitamin E | Antioxidant | 1.0 |
| Vitamin E TPGS | Surfactant | 95.2 |
| OCC | Active agent | 3.8 |

The procedure of Method A was followed.

### Method E. Multi-Component Drug Delivery System

The following ingredients were provided in the amounts indicated.

| Component | Weight (g) | Weight % (w/w) |
|---|---|---|
| Vitamin E | 10.0 | 1.0 |
| Cremophor ELP | 580.4 | 55.9 |
| Labrasol | 89.0 | 8.6 |
| Glycerol Monooleate | 241.0 | 23.2 |
| Ethanol | 80.0 | 7.7 |
| OCC | 38.5 | 3.7 |
| Total | 1038.9 | 100 |

The procedure of Method A was followed.

### Method F. Multi-Component Drug Delivery System

The following ingredients were provided in the amounts indicated and included in a capsule.

| Component | Tradename | Weight % (w/w) |
|---|---|---|
| OCC | FLAVEX Naturextrakte | 0.6 |
| Vitamin E | | 1.3 |
| Caprylocaproyl | Labrasol | 11.1 |
| polyoxyglycerides | Gattefosse 3074TPD | |
| Lauroyl | Gelucire 44/14 | 14.6 |
| polyoxyglycerides | Gattefosse 3061TPD | |
| Polyoxyl 35 Castor | Kolliphor | 72.4 |
| oil | BASF Corp. 50251534 | |
| Total | | 100 |

The procedure of Method A was followed.

### Example 4

### Preparation of Enteric Coated Capsules

### Step I: Preparation of Liquid-Filled Capsule

Hard gelatin capsules (50 counts, 00 size) were filled with a liquid composition of Example 3. These capsules were manually filled with 800 mg of the formulation and then sealed by hand with a 50% ethanol/50% water solution. The capsules were then banded by hand with 22% gelatin solution containing the following ingredients in the amounts indicated.

| Ingredient | Wt. (g) |
|---|---|
| Gelatin | 140.0 |
| Polysorbate 80 | 6.0 |
| Water | 454.0 |
| Total | 650.0 |

The gelatin solution mixed thoroughly and allowed to swell for 1-2 hours. After the swelling period, the solution was covered tightly and placed in a 55° C. oven and allowed to liquefy. Once the entire gelatin solution was liquid, the banding was performed

Using a pointed round 3/0 artist brush, the gelatin solution was painted onto the capsules. Banding kit provided by Shionogi was used. After the banding, the capsules were kept at ambient conditions for 12 hours to allow the band to cure.

### Step II. Coating of Liquid-Filled Capsule

A coating dispersion was prepared from the ingredients listed in the table below.

| Ingredient | Wt. % | Solids % | Solids (g) | g/Batch |
|---|---|---|---|---|
| Eudragit L30D55 | 40.4 | 60.5 | 76.5 | 254.9 |
| TEC | 1.8 | 9.0 | 11.4 | 11.4 |
| AlTalc 500V | 6.1 | 30.5 | 38.5 | 38.5 |
| Water | 51.7 | na | na | 326.2 |
| Total | 100.0 | 100.0 | 126.4 | 631.0 |

If banded capsules according to Step I were used, the dispersion was applied to the capsules to a 20.0 mg/cm² coating level. The following conditions were used to coat the capsules.

| Parameters | Set-up | |
|---|---|---|
| Coating Equipment | Vector LDCS-3 | |
| Batch Size | 500 | g |
| Inlet Air Temp. | 40° | C. |
| Exhaust Air Temp. | 27-30° | C. |
| Inlet Air Volume | 20-25 | CFM |
| Pan Speed | 20 | rpm |
| Pump Speed | 9 rpm (3.5 to 4.0 g/min) | |
| Nozzle Pressure | 15 | psi |
| Nozzle diameter | 1.0 | mm |
| Distance from tablet bed* | 2-3 | in |

*Spray nozzle was set such that both the nozzle and spray path were under the flow path of inlet air.

### Example 5

### Treatment of Pruritus in an Animal—all Dosage Forms

An animal presenting with pruritus is chronically administered OCC according to a prescribed dosing regimen for a period of time. The animal's level of response is determined periodically to determine if the target treatment goal (reduction or elimination of pruritus) has been achieved. If the level of response is too low at one dose, then the dose is escalated according to a predetermined dose escalation schedule until the desired level of therapeutic response in the animal is achieved. Treatment of the animal with OCC composition is continued as needed, and the dose or dosing regimen can be adjusted as needed until the animal reaches the target treatment goal. The animal is maintained on treatment as long as desired or as long as it is prone to exhibit symptoms associated with pruritus.

### Example 6

### Intramuscular Administration of OCC to Dogs

A stock aqueous solution containing ANVIRZEL powder (300 mg; ANVIZEL contained 350-400 microg oleandrin (OLE) per g of powder) dissolved in water for injection (20 mL) was prepared (resulting concentration: 15 mg ANVIRZEL/mL; 17.5-20 microg OLE/mL).

Beagle dogs (16 male, 16 female) ranging in weight from 9.4-11.5 kg (males) and 6.5-10.7 kg (females) were selected for the study. The age of the dogs ranged from 7-11 months on day-1 of administration. Each gender was divided into four cohorts according to the dose (amount of stock solution) administered once daily (for 28 days) via i.m. injection. Water for injection was used as the control.

| | Cohort I | Cohort II | Cohort III | Control |
|---|---|---|---|---|
| ANVIRZEL | 0.375 | 0.75 | 1.5 | 0 |
| (mg/Kg/day) | | | | |
| Equivalent | 0.14 | 0.28 | 0.56 | 0 |
| OLE (microg/ | | | | |
| Kg/day | | | | |
| Volume | 0.025 mL/ | 0.05 mL/ | 0.1 mL/ | 15 mL/ |
| administered | Kg/day | Kg/day | Kg/day | day |

No mortality was observed. No drug-related undesirable side effects were observed.

### Example 7

### Preparation of a Tablet Composition

An initial tabletting mixture of 3% Syloid 244FP and 97% microcrystalline cellulose (MCC) was mixed. Then, an existing batch of composition prepared according to Example 3 was incorporated into the Syloid/MCC mixture via wet granulation. This mixture is labeled “Initial Tabletting Mixture) in the table below. Additional MCC was added extra-granularly to increase compressibility. This addition to the Initial Tabletting Mixture was labeled as “Extra-granular Addition.” The resultant mixture from the extra-granular addition was the same composition as the “Final Tabletting Mixture.”

| Component | Weight (g) | Weight % (w/w) |
|---|---|---|
| Initial Tabletting Mixture | | |
| Microcrystalline cellulose | 48.5 | 74.2 |
| Colloidal Silicon Dioxide/Syloid | 1.5 | 2.3 |
| 244FP | | |
| Formulation from Ex. 3 | 15.351 | 23.5 |
| Total | 65.351 | 100.0 |

### Extragranular Addition

| Component | Weight (g) | Weight % (w/w) |
|---|---|---|
| Initial Tabulating Mixture | 2.5 | 50.0 |
| Microcrystalline cellulose | 2.5 | 50.0 |
| Total | 5 | 100.0 |

### Final Tabletting Mixture:

### Abbreviated

| Component | Weight (g) | Weight % (w/w) |
|---|---|---|
| Microcrystalline cellulose | 4.36 | 87.11 |
| Colloidal Silicon Dioxide/Syloid | 0.06 | 1.15 |
| 244FP | | |
| Formulation from Ex. 3 | 0.59 | 11.75 |
| Total | 5.00 | 100 |

### Final Tabletting Mixture:

### Detailed

| Component | Weight (g) | Weight % (w/w) |
|---|---|---|
| Microcrystalline cellulose | 4.36 | 87.11 |
| Colloidal Silicon Dioxide/Syloid | 0.06 | 1.15 |
| 244FP | | |
| Vitamin E | 0.01 | 0.11 |
| Cremophor ELP | 0.33 | 6.56 |
| Labrasol | 0.05 | 1.01 |
| Glycerol Monooleate | 0.14 | 2.72 |
| Ethanol | 0.05 | 0.90 |
| SCF extract | 0.02 | 0.44 |
| Total | 5.00 | 100.00 |

Syloid 244FP is a colloidal silicon dioxide manufactured by Grace Davison. Colloidal silicon dioxide is commonly used to provide several functions, such as an adsorbant, glidant, and tablet disintegrant. Syloid 244FP was chosen for its ability to adsorb 3 times its weight in oil and for its 5.5 micron particle size.

### Example 8

### HPLC Analysis of Solutions Containing Oleandrin

Samples (oleandrin standard, SCF extract and hot-water extract) were analyzed on HPLC (Waters) using the following conditions: Symmetry C18 column (5.0 μm, 150×4.6 mm I.D.; Waters); Mobile phase of MeOH:water=54:46 (v/v) and flow rate at 1.0 ml/min. Detection wavelength was set at 217 nm. The samples were prepared by dissolving the compound or extract in a fixed amount of HPLC solvent to achieve an approximate target concentration of oleandrin. The retention time of oleandrin can be determined by using an internal standard. The concentration of oleandrin can be determined/calibrated by developing a signal response curve using the internal standard.

### Example 9

### Preparation of Veterinary Pharmaceutical Compositions

A pharmaceutical composition of the invention can be prepared according to any of the following methods. Mixing can be done under wet or dry conditions. The pharmaceutical composition can be compacted, dried or both during preparation. The pharmaceutical composition can be portioned into any of the dosage forms described in this disclosure.

### Method A.

At least one pharmaceutical excipient is mixed with at least one OCC disclosed herein.

### Method B.

At least one pharmaceutical excipient is mixed with oleandrin.

### Method C.

At least one pharmaceutical excipient is mixed with an extract containing oleandrin.

### Method D.

At least one pharmaceutical excipient is mixed with a combination of two or more extracts containing oleandrin.

### Method E.

At least one pharmaceutical excipient is mixed with any of the compositions of Methods A-D and at least one triterpene. The triterpenes are selected from the group consisting of oleanolic acid, ursolic acid, and betulinic acid.

### Method F.

At least one pharmaceutical excipient is mixed with any of the compositions of Methods A-E and at least one compound selected from the group consisting of oleandrigenin, desacetyl-oleandrin, gitoxigenin, kanerocin, kanerodione, Nerium F, neritaloside, odoroside, adynerin, and odoroside-G-acetate.

The veterinary composition of any one of methods A-F can further comprise polyphenol(s), carbohydrate(s), flavonoid(s), amino acid(s), soluble protein(s), cellulose, starch, alkaloid(s), saponin(s), tannin(s), or a combination thereof.

### Example 10

### Preparation of Triterpene Mixtures

The following compositions were made by mixing the specified triterpenes in the approximate molar ratios indicated.

| | Triterpene (Approximate Relative Molar Content) | | |
|---|---|---|---|
| Composition | Oleanolic acid (O) | Ursolic acid (U) | Betulinic acid (B) |
| I (A-C) | 3 | 2.2 | 1 |
| II (A-C) | 7.8 | 7.4 | 1 |
| III (A-C) | 10 | 1 | 1 |
| IV (A-C) | 1 | 10 | 1 |
| V (A-C) | 1 | 1 | 10 |
| VI (A-C) | 1 | 1 | 0 |
| VII (A-C) | 1 | 1 | 1 |
| VIII (A-C) | 10 | 1 | 0 |
| IX (A-C) | 1 | 10 | 0 |

For each composition, three different respective solutions were made, whereby the total concentration of triterpenes in each solution was approximately 9 μM, 18 μM, or 36 μM.

| Composition | Triterpene (Approximate Content of Each, μM) | | |
|---|---|---|---|
| (total triterpene | Oleanolic acid | Ursolic acid | Betulinic acid |
| content, μM) | (O) | (U) | (B) |
| I-A (36) | 17.4 | 12.8 | 5.8 |
| I-B (18) | 8.7 | 6.4 | 2.9 |
| I-C (9) | 4.4 | 3.2 | 1.5 |
| II-A (36) | 17.3 | 16.4 | 2.2 |
| II-B (18) | 8.7 | 8.2 | 1.1 |
| II-C (9) | 4.3 | 4.1 | 0.6 |
| III-A (36) | 30 | 3 | 3 |
| III-B (18) | 15 | 1.5 | 1.5 |
| III-C (9) | 7.5 | 0.75 | 0.75 |
| IV-A (36) | 3 | 30 | 3 |
| IV-B (18) | 1.5 | 15 | 1.5 |
| IV-C (9) | 0.75 | 7.5 | 0.75 |
| V-A (36) | 3 | 3 | 30 |
| V-B (18) | 1.5 | 1.5 | 15 |
| V-C (9) | 0.75 | 0.75 | 7.5 |
| VI-A (36) | 18 | 18 | 0 |
| VI-B (18) | 9 | 9 | 0 |
| VI-C (9) | 4.5 | 4.5 | 0 |
| VII-A (36) | 12 | 12 | 12 |
| VII-B (18) | 6 | 6 | 6 |
| VII-C (9) | 3 | 3 | 3 |
| VIII-A (36) | 32.7 | 3.3 | 0 |
| VIII-B (18) | 16.35 | 1.65 | 0 |
| VIII-C (9) | 8.2 | 0.8 | 0 |
| IX-A (36) | 3.3 | 32.7 | 0 |
| IX-B (18) | 1.65 | 16.35 | 0 |
| IX-C (9) | 0.8 | 8.2 | 0 |

### Example 11

### Preparation of Topical Composition

Topical aqueous liquid compositions were prepared by mixing the following components in the approximate amounts indicated. The compositions were included in bottles or spray bottles intended for multi-use, e.g. an amount sufficient to provide doses for a treatment period of one week up to three months.

| Ingredient | Content |
|---|---|
| water | 55-60% (q.s) |
| glycerin | 15-25% |
| polysorbate 20 | 5-10% |
| propylene glycol | 4-6% |
| hydrolzed collagen | 0.5-5% |
| aloe vera 200x | 0.5-5% |
| tocopherol (vitamin e) | 0.5-3% |
| pramoxine | 0.5-2% |
| sodium PCA | 0.5-5% |
| caprylyl glycol, hexylene glycol, phenoxyethanol | 0.5-2% |
| Oleandrin | 0.01-10 μg/mL |
| | or 0.1-5 μg/mL |
| allantoin | 0.05-0.5% |
| chamomile oil | 0.01-0.05% |
| clove essential oil | 0.01-0.05% |
| Dimethicone | 0.05-2% |

When included in a spray bottle, the amount of fluid per spray varied from 0.25 mL to 2.5 mL.

### Example 12

### Treatment of Pruritus in a Dog (Oral)

Dogs or cats presenting with pruritus are administered oleandrin containing composition to treat pruritus.

Method A. Oral administration

Dogs or cats are administered an oral OCC as described herein, in particular of the formulations containing any of the following extracts: supercritical fluid (SCF) extract, ethanolic (EtOH) extract, aqueous ethanolic (wEtOH) extract, methanolic (MeOH) extract, water (W) extract, subcritical fluid (SbCF) extract, and combinations thereof. Specific extracts were selected from PBI-05204 (SCF), PBI-01220 (EtOH), PBI-1× (wEtOH), PBI-06150 (SbCF+EtOH), PBI-01207 (W).

The animal is orally administered 1-4 doses per day of one or more of the above compositions according to a prescribed dosing regimen for a period of time. The animal's level of clinical response is determined periodically. The level of therapeutic response can be determined by determining the animal's reduction or elimination of pruritus. If the level of clinical response is too low at one dose, then the dose is escalated according to a predetermined dose escalation schedule until the desired level of clinical response in the animal is achieved. Chronic daily treatment of the animal with the composition is continued as needed and the dose or dosing regimen can be adjusted as needed until the animal reaches the desired clinical endpoint.

### Example 13

### In Vitro Evaluation of Impact of Oleandrin and Extract Upon Pruritus Related Cytokines and Chemokines

Reagents. Dulbecco's phosphate-buffered saline (Gibco cat. #141190-136), lipopolysaccharide (LPS) (Invitrogen cat. #00-4976-93), Roswell Park Memorial Institute 1640 cell culture medium (Gibco cat. #11835-030), Fetal Bovine Serum (Gibco cat. #A38401-01), penicillin-streptomycin 100× (Gibco cat. #15140-122), CD69 fluorescein isothiocyanate (clone FN50, Invitrogen cat. #11-0699-42), CD56 phycoerythrin (clone CMSSB Invitrogen cat. #12-0567-42), and CD3 Super Bright 645 (clone OKT3 Invitrogen cat. #64-0037-42) were purchased from Thermo Fisher Scientific (Waltham, MA, USA). CD25 Brilliant Violet 421 (clone 2A3 BD cat. #564033) and sodium heparin Vacutainer tubes (BD cat. #367878) were purchased from Becton-Dickinson (Franklin Lakes, NJ, USA). The human erythroleukemia cell line K562 was purchased from American Type Culture Collection (Manassas, VA, USA). Customized Bio-Plex Pro™ human cytokine arrays were purchased from Bio-Rad Laboratories Inc. (Hercules, CA, USA). Oleandrin (Sigma cat. #06069-5MG) and Interleukin-2 (IL-2) (Sigma cat. #17908-10KU) were purchased from Sigma-Aldrich Co. (St Louis, MO, USA). Lympholyte Poly (Cedarlane cat. #CL5070) was purchased from CedarLane (Burlington, NC, USA)

Test products. PBI-05204 (PBI) is a supercritical CO₂ extract of Nerium oleander leaves. It was provided by Phoenix Biotechnology, Inc. (San Antonio, TX, USA). Characterization of PBI-05204 was carried out using AccuTOF-DART mass spectrometer (Jeol UAS, Peabody, MA, USA). This batch of extract contained oleandrin (1.74% by wt) and other compounds obtained from the plant material.

The two products were handled in the following manner: (1) A stock solution of PBI was prepared in DMSO, and subsequent dilutions were prepared in phosphate-buffered saline. The amount of PBI employed in the assays was normalized based upon its oleandrin content. (2) A stock solution of 5 mg/mL oleandrin was prepared in DMSO, and dilutions were prepared in phosphate-buffered saline. The range of concentrations in cell cultures was calculated based on the % oleandrin and ranged from 0.5 to 500 ng oleandrin/mL cell culture medium. Matching concentrations of DMSO served as the solvent control.

Immune cell activation. Peripheral venous blood was drawn from healthy human donors upon written informed consent, as approval by the Sky Lakes Medical Center Institutional Review Board, Federalwide Assurance 2603. The blood was drawn into heparin vacutainer vials, and the peripheral blood mononuclear cells (PBMC) were isolated using Lympholyte Poly by centrifugation for 35 min at 400×g. The PBMC were washed twice in PBS and counted, and the density was adjusted to establish 0.18 mL cultures with a cell density at 106/mL, using Roswell Park Memorial Institute 1640 medium containing 10% fetal calf serum and 1% penicillin-streptomycin.

Serial dilutions of products or LPS (10 ng/mL) were added to cultures at a volume of 20 μL, so each culture well had a final volume of 0.2 mL/well. Cultures were then incubated at 37° C., 5% CO₂ for 24 h. The highly inflammatory LPS from Escherichia coli was used as a positive control for immune-cell activation. In parallel, IL-2 was used as a positive control for natural killer (NK)-cell activation, at a concentration of 100 IU/mL in cell culture. Untreated negative control cultures consisted of PBMC exposed to phosphate-buffered saline in the absence of test products. All treatments, including each concentration of test product and each positive and negative control, were tested in triplicate. After 24 h, blood cells were isolated from each culture well and stained for 15 min with fluorochrome-labeled antibodies at the recommended concentration. PBMCs were then fixed in formalin (%). The fluorescence intensities for CD3, CD25, CD56, and CD69 were measured by flow cytometry, using an Attune NxT acoustic-focusing flow cytometer (Thermo Fisher Scientific, Waltham, MA, USA). Data analysis utilized gating on forward scatter and side scatter to create electronic gates for the lymphocyte and monocyte/macrophage populations. The lymphocyte subpopulation was further analyzed for CD25 and CD69 expression on CD3−CD56+ NK cells, CD3+ CD56+ NKT cells, and CD3+CD56− T cells.

Production of cytokines, chemokines, and growth factors. After 2, 8, and 24 h of incubation, the supernatants were harvested from the PBMC cultures described above. Levels of 27 cytokines and chemokines were quantified using Bio-Plex protein arrays (Bio-Rad Laboratories Inc., Hercules, CA, USA) and utilizing xMAP technology (Luminex, Austin, TX, USA). The cytokine array included: IL-1β, IL-1ra, IL-2, IL-4, IL-5, IL-6, IL-7, IL-8, IL-9, IL-10, IL-12 (p70), IL-13, IL-15, IL-17, IL-21, IL-22, IL-23, IL-25, IL-31, IL-33, Eotaxin, basic FGF, G-CSF, GM-CSF, IFN-γ, IP-10, MCP-1 (MCAF), MIP-1α, MIP-1β, PDGF-BB, RANTES, TNF-α, and VEGF.

Natural killer (NK) cell cytotoxicity towards K562 human myelogenous leukemia target cells. The cell surface expression of CD107a (LAMP-1) is a marker for secretory degranulation of NK and activated cytotoxic T cells when they are co-cultured with malignant or virus-infected target cells. Peripheral blood mononuclear cells from healthy human donors were co-cultured with K562 cells in the absence versus presence of test products or matching concentrations of the DMSO solvent control. All treatments, including each concentration of test product and each positive and negative control, were tested in triplicate. CD107a-FITC antibody (6.5 μL/well) was added at the beginning of each culture, along with the protein transport inhibitor BD GolgiStop™, which contains Monensin and blocks intracellular protein transport processes. After 4 h of incubation at 37° C., 5% CO₂, the cells were washed in phosphate-buffered saline and stained with CD3-PerCP and CD56-PE antibodies and then fixed in formalin. Flow cytometry analysis was performed where the strategy involved initial gating of the lymphocyte and monocyte subpopulations using the forward/side scatter plot, followed by analysis of T cells and NK cells in the lymphocyte population. The fluorescence intensity of CD107a was evaluated as the mean fluorescence intensity on monocytes, NK cells, and T cells.

### Example 14

### Treatment of Pruritus in an Animal (Buccal/Sublingual)

### Method A. Administration of Oleandrin-Containing Aqueous Solution

A dog presenting with pruritus was administered the aqueous liquid composition of Example 2. It contained 0.1-5 μg OLE/mL. The composition (0.25-1 mL) was administered (1-4 times daily) directly to the dog's mouth, thereby resulting in a combination of buccal and sublingual administration. The dog exhibited relief from the itching. Both inflammation and itchiness were relieved. Daily administration 1-4 times per day was continued and the dog exhibited no return of the pruritus and no composition-related side effects.

### Method B. Administration of Oleandrin-Containing Oil-Based Solution

A dog presenting with pruritus was administered the MCT-based composition of Example 21. It contained 12-25 μg OLE/mL. A few drops of the composition (0.25-1 mL) is administered (1-4 times daily) directly to the dog's mouth, thereby resulting in a combination of buccal and sublingual administration. The dog exhibited relief from the itching. Both inflammation and itchiness were relieved. Daily administration 1-4 times per day is continued and the dog exhibits no return of the pruritus and no composition-related side effects.

### Example 15

### Treatment of Pruritus in an Animal (Topical)

A dog presenting with pruritus was administered the aqueous liquid composition of Example 11. The composition was sprayed (1-4 pumps) directly onto the itchy area of the dog and rubbed into the skin and fur/hair. The dog exhibited almost immediate relief from the itching. Both inflammation and itchiness were relieved. Daily administration 2-4 times per day was continued, and the dog exhibited no return of the pruritus and no composition-related side effects.

### Example 16

### Statistical Analysis

The statistical significance of experimental data sets was determined using unpaired two-tailed Student's t-tests (alpha=0.05) and calculated P-values using the Shapiro-Wilk normality test and Graphpad Prism 7.03 software. The P-values were defined as: 0.1234 (ns), 0.0332 (*), 0.0021 (**), 0.0002 (***), <0.0001 (****). Unless otherwise noted, error bars represent the SEM from at least three independent experiments.

### Example 17

### Preparation of Aqueous Ethanolic Extract of Nerium Oleander

The purpose of this was to prepare an ethanolic extract by extraction of Nerium oleander biomass with aqueous ethanol.

Ground dried leaves were repeatedly treated with aqueous ethanol (60-70% v/v ethanol; 40-35% v/v water). In some cases, the temperature was above ambient, e.g. hot (40° C. to refluxing). The combined ethanolic supernatants were combined and filtered and then concentrated by evaporation in vacuo to reduce the amount of ethanol and water therein (by about 10-fold) and provide crude ethanolic extract comprising about 2-4 mg of oleandrin/mL of extract (which has about 50% v/v ethanol content).

### Example 18

### In Vitro Evaluation of Oleandrin Toxicity Against Cells

The purpose of this assay was to determine the relative potential toxicity of oleandrin against various cells in vitro.

Oleandrin (PhytoLab, Vestenbergsgreuth, Germany) was dissolved at a concentration of 1 mg/ml in DMSO. The desired concentration range to be used in cytotoxicity testing is 0.005-1 ug/ml in 0.0005-0.1% wt in DMSO, respectively. Lactate dehydrogenase release assay (LDH assay) was be used to determine the cytotoxic effect of different concentration of Oleandrin on different cell cultures. LDH is a cytosolic enzyme that is released only from damaged cells (due to increased membrane permeability) to the outside medium that will convert the lactate in the medium into pyruvate in a coupled reaction that includes the reduction of NAD+ into NADH, the latter is oxidized back to NAD+ in the presence of Diaphorase in the LDH kit mix that leads to the reduction of water soluble tetrazolium (INT), that is also in the kit mix, into red Formazon product that can be read by an ELISA reader at a wave length of 490 nm. The cytotoxic effect of 8 different concentrations of Oleandrin (0.005-1 ug/ml) on 3 different cell line was tested on BT (Bos taurus turbinate), MDBK (Bos taurus kidney) and MARC 145 (monkey kidney) cells in triplicates at 2 different times points 24 and 48 hours post treatment.

This was achieved by preparing different concentration of cells in 100 μl volume staring at 1000 cell/100 μl to 20,000 cell/100 μl in a 2-fold serial dilution manner. This serial dilution was done in triplicate for 2 sets of cells, one set used as cell control, that would report the spontaneous LDH release, and the other set was treated with cell lysis buffer to report the maximum LDH release. After performing the test according to the manufacturer instructions, the average OD value reading of the triplicate for each dilution in each set was taken and plotted against number of cells. The best cell seeding capacity/100 μl volume was the one that achieved maximum LDH release of 1.6-2 and spontaneous LDH release of less than 0.5. after 30 min of incubation with the kit mix (manufacturer instructions) at both time points. The optimal number of cells/well in 100 μL of growth medium (as determined in preliminary experiments) was plated in triplicate in wells in a 96-well tissue culture plate. Cells were incubated overnight at 37° C. with the appropriate level of CO₂. The following day the growth medium was removed by washing the cells twice with PBS. The growth media was replaced with 100 μl of maintenance media containing either 1-0.005 ug/ml oleandrin, 0.1-0.0005% DMSO without drug, or untreated media to serve as controls for the maximum and spontaneous release 250 of LDH. All treatments were added to triplicate wells, and the plate was returned to the 37° C./5% CO₂ incubator for 24-48 hours. At either 24 or 48 hours post-treatment, the plate was removed from the incubator and the LDH released into the supernatant was assessed by CyQUANT LDH toxicity assay (Thermofisher, Eugene, OR) according to the manufacturer's directions. Absorbance is measured at 490 nm and 680 nm using Spectramax i3x. The corrected OD value of the max LDH release control should be around 1.6-2 and that for the spontaneous LDH release control should be below 0.5.

To determine the cytotoxicity of Oleandrin and DMSO, the following equation was applied to the corrected OD value of each concentration:

% cytotoxicity of individual concentration=corrected OD value of ((Treatment*−Spontaneous LDH release)/(Maximum LDH release−Spontaneous LDH release))×100

To determine the safe dosage of Oleandrin, the % cytotoxicity was maintained as less than 2% in each time points for the Oleandrin concentration and the corresponding DMSO concentration.

### Example 19

### Preparation of Subcritical Fluid Extract of Nerium Oleander

An improved process for the preparation of an oleandrin-containing extract was developed by employing subcritical liquid extraction rather than supercritical fluid extraction of Nerium oleander biomass.

Dried and powdered biomass was placed in an extraction chamber, which was then sealed. Carbon dioxide (about 95% wt) and alcohol (about 5% wt; methanol or ethanol) were injected into the chamber. The interior temperature and pressure of the chamber were such that the extraction medium was maintained in the subcritical liquid phase, rather than the supercritical fluid phase, for a majority or substantially all of the extraction time period: temperature in the range of about 2° C. to about 16° C. (about 7° C. to about 8° C.), and pressure in the range of about 115 to about 135 bar (about 124 bar). The extraction period was about 4 h to about 12 h (about 6 to about 10 h). The extraction milieu was then filtered and the supernatant collected. The carbon dioxide was vented from the supernatant, and the resulting crude extract was diluted into ethanol (about 9 parts ethanol:about 1 part extract) and frozen at about −50° C. for at least 12 h. The solution was thawed and filtered (100 micron pore size filter). The filtrate was concentrated to about 10% of its original volume and then sterile filtered (0.2 micron pore size filter). The concentrated extract was then diluted with 50% aqueous ethanol to a concentration of about 1.5 mg of extract per mL of solution.

The resulting subcritical liquid (SbCL) extract comprised oleandrin and one or more other compounds extractable from Nerium oleander, said one or more other compounds being as defined herein.

### Example 20

### Preparation of Ethanolic Extract of Nerium Oleander

The purpose of this was to prepare an ethanolic extract by extraction of Nerium oleander biomass with aqueous ethanol.

Ground dried leaves were repeatedly treated with aqueous ethanol (90-95% v/v ethanol; 10-5% v/v water). In some cases, the temperature was above ambient. The combined ethanolic supernatants were combined and filtered and then concentrated by evaporation in vacuo to reduce the amount of ethanol and water therein and provide crude ethanolic extract comprising about 25 mg of oleandrin/mL of extract (which has about 50% v/v ethanol content).

### Example 21

### Preparation of Dosage Form Comprising a Combination of Extracts of Nerium Oleander

The purpose of this was to prepare a dosage form (OCC, PBI-06150) containing a portion (1 wt %) of the ethanolic extract of Example 19 combined with a portion (1 wt %) of the SbCL extract of Example 20, medium chain triglyceride (95 wt %), and flavoring agent (3 wt %).

### Example 22

### Preparation of Aqueous Methanolic Extract of Nerium Oleander

The purpose of this was to prepare a methanolic extract by extraction of Nerium oleander biomass with methanol.

Ground dried leaves were repeatedly treated with methanol. In some cases, the temperature was above ambient, e.g. hot (>21 C to refluxing). The combined methanolic supernatants were combined and filtered and then concentrated by evaporation in vacuo to substantially to dryness and provide crude methanolic extract comprising about 2-4 mg of oleandrin/g of solid extract.

### Example 23

### Preparation of Foodstuff Containing Oleandrin (Oleander Extract)

The purpose of this was to prepare an edible product comprising oleandrin or comprising an oleandrin-containing extract, in any suitable form.

An extract of Nerium oleander is prepared. Its solvent can be removed to either concentrate the extract or to remove the solvent completely. The resulting concentrate is mixed with one or more edible materials suitable for animals. The foodstuff can be any treat or feed or liquid known to be suitable for consumption by animals, in particular dogs or cats.

### Example 24

### Administration of Oleandrin Containing Composition to Dogs (Oral)

The SCF extract (PBI-05204) was formulated as detailed above into a micelle forming delivery system, i.e. a composition that forms micelles when the composition is mixed with aqueous, such as water, buffer, saliva, gastric fluid, intestinal fluid, or colonic fluid. The content of OLE in the formulated composition was typically in the range of 0.05-0.1%.

Formulated PBI-05204 composition or control vehicle were orally administered to beagle dogs at once daily doses of 0.1, 0.3, or 1.0/0.6 mg/kg oleander extract (equivalent to 2.3 μg, 6.9 μg, and 23/13.8 μg of oleandrin/kg, respectively) for 28 consecutive days followed by a 14-day recovery period. This resulted in a no-observable-adverse-effect-level (NOAEL) of 0.1 mg/kg. The adverse effects at 0.3 or 1.0/0.6 mg/kg were reversible following a 14-day recovery period.

In order to determine an approximate MTD, a dose-range finding (single dose) study of formulated PBI-05204 composition was conducted in beagle dogs. The relative drug toxicity in dogs treated with escalating oral doses was determined. One male and one female dog were provided the following escalating doses of 5, 10, and 20 mg/kg (equivalent to 115 μg, 230 μg, and 460 μg of oleandrin/Kg, respectively) followed by a 3-day washout/observation period between doses. The results in the male indicated a dose-dependent increase in abnormal clinical observations consisting of vomiting, abnormal stool, increased salivation, labored breathing, decreased activity, and death. After the female dog received 20 mg/kg of formulated PBI-05204 (equivalent to 460 μg of oleandrin/Kg), a severe arrhythmia characterized as sinus arrest with ventricular escape rhythm.

A 28-day toxicity study of formulated PBI-05204 in beagle dogs followed by a 14-day recovery period was conducted. The objective of this study was to determine the potential systemic toxicity of test article administered once daily for 28 consecutive days via oral (gavage) administration followed by a 14-day recovery period. A total of 36 dogs were dosed with test article PBI-05204 containing 37.2 mg/mL (at concentrations of 0.1, 0.3, or 1.0/0.6 mg/kg) oleander extract (equivalent to 2.3, 6.9, 23/13.8 ug oleandrin/kg, respectively) or control vehicle PBI-05204 Control Vehicle (Placebo) via oral gavage once daily for 28 consecutive days (except for Group 4 animals). There were no early deaths during the study. All animals survived until their scheduled necropsy date. Test article-related clinical findings include the following: abnormal stool primarily in Groups 3 and 4 males, thin body only in Group 4 animals with a delayed onset in females when compared to males, decreased activity in Group 4 males only, inappetence in Group 4 males only, defecation decreased in Group 4 males only, predominant post-dose vomiting in Group 4 (males>females), predominant increased salivation in Group 4 (males>females), bloodshot eyes in Group 4 males only, predominant excessive lacrimation in Group 4 animals, and ptosis in Group 4 males only. Compared to Study Day 1, Study Day 29 bodyweights for Groups 3 and 4 males were decreased. There were no apparent test article-affects on feed consumption with the exception of decreased feed intake in Group 4 males on Study Days 7 through 13. There were no apparent test article-affects on individual body temperatures with the exception of decreased body temperatures in Group 4 males on Study Day 8. Based on the measured hematology parameters, there were no apparent biological or toxicological findings. On Study Day 29, Group 4 male heart weight trends (absolute and relative) were decreased in comparison to concurrent controls. This finding appears to be test article-related. The administration of test article did not cause a discernable change in QRS duration, or QT interval, or QTcf or QTcv throughout this study. However, in dogs receiving 1.0/0.6 mg/kg/day, the heart rate was decreased. In dogs receiving 0.3 or 1.0/0.6 mg/kg/day, atrioventricular conduction was slowed and indicated by prolongation of the PR interval and the development of secondary degree atrioventricular block. Also atrial premature depolarization developed in dogs receiving over 0.3 or 1.0/0.6 mg/kg/day. The presence of these findings both before and after test article-administration on Study Week 4 suggests at least a 23 hour duration of the test article-induced changes. In conclusion, the administration of 0.1; 0.3; or 1.0/0.6 mg/kg) oleander extract, or control vehicle PBI-05204 Control Vehicle (Placebo) via oral gavage once daily for 28 consecutive days (except for Group 4 animals) followed by a 14 day recovery period resulted in a no-observable-adverse-effect-level (NOAEL) of 0.1 mg/kg (equivalent to 2.3 ug oleandrin/kg). The adverse effects at 0.3 or 1.0/0.6 mg/kg were reversible following a 14-day recovery period.

### Example 25

### Treatment of Pruritus in a Human (Topical)

The topical formulation of Example 11 containing oleandrin (0.01-10 microg/mL) as the cardiac glycoside was sprayed (volume of about 0.1-1 mL) onto a patch of itchy skin one to four times per day. Relief from itching occurred in less than about 10 min.

### Example 26

### Comparative Evaluation of Oleander Extract, Oleandrin, and Oclacitinib in Cytokine Assay

Oleandrin (Sigma-Aldrich), oclacitinib (Sigma-Aldrich), and an oleander extract (PBI 05204 from Phoenix Biotechnology, Inc) were prepared in DMSO then diluted in phosphate-buffered saline such that the highest concentration in the cell-based assays was 500 ng/mL. PBI-05204 is a supercritical CO₂ extract of Nerium oleander leaves and was provided by Phoenix Biotechnology, Inc. (San Antonio, Texas). Characterization of PBI-05204 was carried out using an AccuTOF-DART mass spectrometer (Jeol UAS, Peabody, MA). Specific content of the extract was previously reported. The extract contained cardiac glycosides, oleandrin (2.99%) and oleandrigenin (3.31%); triterpenoid acids, ursolic and betulinic acids (combined total of 15.29%), oleanolic acid (0.60%), odoroside (0.8%); Urs-12-ene-3β, 28-diol/botulin (5.44%), 3β, 3β-hydroxy-12-olean en-28-oic acid (14.26%); 28-nours-12-en-3β-ol (4.94%); and urs-12-en-30-ol (4.76%). The concentration of OE was normalized according to its oleandrin content. The concentration in cell cultures was determined based on literature and initial viability testing using a standard MTT assay protocol.

We tested three different concentrations of oclacitinib: 10 nM, 500 nM, and 10 μM, based on published data. DMSO was used as a control. Anti-inflammatory properties of oclacitinib have been reported at concentrations 36-249 nM, and inhibition of T cell proliferation was seen at 10 μM. One study found that oclacitinib inhibited JAK kinases at varying concentrations, with IC₅₀ ranging from 10-99 nM, with the most potent inhibition of AK1 kinase at 10 nM. Testing for cellular relative metabolic activity was conducted prior to cytokine testing, to ensure that the cells were viable and active when treated with test products.

Two types of cells were used: 1) primary canine dermal fibroblasts (Cell Biologics, Chicago, IL, USA), and 2) the DH82 macrophage canine cell line (Distributor: Millipore Sigma, St. Louis, MO, USA; Origin: Culture Collections UK Health Security Agency, Porton Down, Salisbury, UK). The DH82 cell line has been shown to be a useful tool in evaluating inflammatory reactivity in canine macrophages, with similarities in phenotype and function to primary canine monocyte-derived macrophages.⁹ DH82 cells have been used to study macrophage polarization towards M1 versus M2 subtypes relevant for allergic reactivity, as the M2a subtype of macrophages plays an important role in immunoglobulin E (IgE-mediated allergies) and other Th2 type immune reactions.

The test products were compared in a selected panel of lab assays to expand knowledge of their anti-inflammatory activities as it pertains to allergies. Both types of cell cultures were tested under normal or inflamed conditions with use of lipopolysaccharide (LPS, Sigma-Aldrich, Inc., St. Louis, MO, USA). Culture supernatants were collected after 24 hours, and used for testing of cytokine levels using Bio-Plex protein arrays (Bio-Rad Laboratories Inc.) and utilizing xMAP technology (Luminex, Austin, TX, USA). The following cytokine panels were used: 1) Human 15-plex for Th17 cytokines: IL-1β, IL-4, IL-6, IL-10, IL-17A, IL-17F, IL-21, IL-22, IL23, IL-25, IL-31, IL-33, IFN-γ, sCD40L, TNF-α, 2) Canine 11-plex: IL-2, IL-6, IL-8, TGF-β1, IL-12/IL-23p40, NGF-0, IFN-γ, TNF-α, MCP-1 (CCL2), VEGF-A, and SCF. The use of a human cytokine multiplex panel of antibodies in addition to a canine cytokine multiplex panel was necessary in order to try to determine the broadest array of cytokine response data relevant to atopic dermatitis benefiting from the antibody cross-reactivity between human and canine cytokines.

### Example 27

### Preparation of Dog Chews Containing Oleandrin (Oleander Extract)

Dog chews containing oleandrin in the form of oleander extract were prepared by mixing grain flour, bran, peanut flour, peanut butter, peanut oil, water, and oleander extract. The ingredients were thoroughly mixed, and the chews were made by extrusion of the mass followed by cutting of the extruded mass to predetermined weights. The average size of the chews was 2.5 g, 5.0 g, and 7.5 g, each containing an average of 30 microg oleandrin, 60 microg oleandrin, and 90 microg oleandrin, respectively.

### Example 28

### Treatment of Atopic Dermatitis (Pruritus) in Dogs by Administration of Oleandrin (Oleander Extract)

Oleandrin (oleander extract) was orally administered in two different forms. The oil-based formulation of Example 21 was sprayed onto dog treats and administered between meals or sprayed onto dog food and administered as part of a meal. The chews of Example 27 were orally administered between meals. Both products were administered 1-2 times daily at doses ranging from 2-6 (preferably 3-5) microg oleandrin/Kg of bodyweight. The oil-based formulation of Example 21 was also topically administered 1-2 times daily by applying the formulation directly to the animal's skin as needed.

As used herein, the terms “about” or “approximately” are taken to mean ±10%, ±5%, ±2.5% or ±1% of a specified valued. As used herein, the term “substantially” is taken to mean “to a large degree” or “at least a majority of” or “more than 50% of”

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

1. A method of treating pruritus in a subject with pruritus or preventing pruritus in a subject at risk of developing pruritus or prone to pruritus, the method comprising administering to a subject one or more doses of a cardiac glycoside-containing composition (CGCC),
wherein the subject is an animal or human.

2. (canceled)

3. The method of claim 1 further comprising,
administering to the subject one or more doses of an oclacitinib, or salt thereof containing composition,
wherein the CGCC and oclacitinib, or salt thereof, containing composition are administered:
a) in the same composition or in separate compositions;
b) via the same route of administration or via different routes of administration;
c) simultaneously, sequentially, or in an overlapping manner;
d) with overlapping dosing periods or separate dosing periods; or
e) a combination thereof.

4. The method of claim 3, wherein:
a) the CGCC and oclacitinib, or salt thereof, containing composition are both administered systemically;
b) the CGCC is administered non-systemically, and the oclacitinib, or salt thereof, containing composition is administered systemically;
c) the CGCC is administered systemically, and the oclacitinib, or salt thereof, containing composition is administered non-systemically; or
d) the CGCC is administered orally, buccally, sublingually, topically, or a combination thereof, and the oclacitinib, or salt thereof, containing composition is administered orally, buccally, sublingually, an/er topically, or a combination thereof.

5. The method of claim 1, wherein the CGCC is administered topically, buccally, sublingually, or orally.

6. The method of claim 1, wherein the animal is a dog, cat, cow, or horse, and further wherein the CGCC is included in a feed and/or liquid administered orally to the animal.

7. The method of claim 1, wherein the one or more doses of the CGCC is about 0.05 to about 0.5 μg/kg/day, based upon a unit amount of cardiac glycoside per kg of bodyweight per day.

8. The method of claim 1, wherein following administration of the one or more doses, the plasma concentration of cardiac glycoside in the animal is in the range of about 0.005 ng/mL to about 10 ng/mL.

9. The method of claim 1, wherein the one or more doses are administered:
a) one or more times daily;
b) at least three days per week;
c) at least five days per week; or
d) every day of the week, and
further wherein the one or more doses are administered to a subject with pruritus until the pruritus is decreased or eliminated.

10. The method of claim 7, wherein the one or more doses are administered for a treatment period of one or more weeks or one or more months.

11. (canceled)

12. The method of claim 1, wherein the cardiac glycoside comprises a) oleandrin; b) digoxin; or c) a combination of oleandrin and digoxin.

13-14. (canceled)

15. The method of claim 3, further comprising one or more excipients, and
wherein the cardiac glycoside is oleandrin and the oleandrin is present in an amount of about 0.05-100 μg, and wherein the oclacitinib is present in an amount of about 0.5-50 mg.

16. The method of claim 1, wherein the CGCC further comprises one or more of the following:
a) one or more compounds extracted from Nerium oleander plant material;
b) one or more triterpenoic acids;
c) one or more topical anesthetics;
d) one or more emollients;
e) one or more antioxidants;
f) one or more solvents;
g) one or more oils;
h) allantoin;
i) oclacitinib, optionally present as the maleate salt;
j) one or more active polysaccharides from aloe vera, the polysaccharides being selected from the group consisting of glucomannan, polymannose, acemannan, and 1-4)-acetylated polymannose;
k) colloidal oatmeal;
l) one or more preservatives;
m) one or more surfactants;
n) one or more anti-foaming agents;
o) one or more lubricants;
p) one or more corticosteroids;
q) collagen, e.g. hydrolyzed collagen;
r) one or more carboxylic acids;
s) one or more moisturizers;
t) one or more amino acids;
u) one or more humectants;
v) one or more buffering agents;
w) one or more topical skin cleansers;
x) one or more antihistamines;
y) cyclosporine;
z) one or more skin permeation enhancers; or
aa) C-mAB-containing composition.

17. (canceled)

18. The method of claim 1, wherein the CGCC further comprises:
a) oleanolic acid as a free acid, salt, or prodrug and ursolic acid as a free acid, salt, or prodrug;
b) oleanolic acid as a free acid, salt, or prodrug and betulinic acid as a free acid, salt, or prodrug;
c) oleanolic acid as a free acid, salt, or prodrug, ursolic acid as a free acid, salt, or prodrug, and betulinic acid as a free acid, salt, or prodrug;
d) at least two triterpenes selected from the group consisting of oleanolic acid as a free acid, salt, or prodrug, ursolic acid as a free acid, salt, or prodrug, betulinic acid as a free acid, salt, or prodrug; or
e) one or more of oleanolic acid, ursolic acid, betulinic acid, kanerocin, kanerodione, oleandrigenin, desacetyl-oleandrin, gitoxigenin, Nerium F, neritaloside, odoroside, adynerin, or odoroside-G-acetate.

19. The method of claim 1, wherein the CGCC further comprises polyphenol(s), carbohydrate(s), flavonoid(s), amino acid(s), soluble protein(s), cellulose, starch, alkaloid(s), saponin(s), tannin(s), or any combination thereof.

20. The method of claim 1, wherein the CGCC composition comprises an extract of biomass, and wherein the biomass is plant material from Nerium species of Agrobacterium species biomass.

21-23. (canceled)

24. The method of claim 20, wherein the extract further comprises one or more cardiac glycoside precursors, one or more glycone constituents of cardiac glycosides, or a combination thereof.

25. (canceled)

26. The method of claim 1, wherein the CGCC further comprises at least one inhibitor that reduces or inhibits the rate of metabolism or digestion of the cardiac glycoside, thereby increasing the plasma concentration half-life of the cardiac glycoside in the subject.

27-36. (canceled)

37. The method of claim 1, wherein the CGCC aids with one or more of:
a) maintaining healthy skin;
b) maintaining a healthy coat in an animal;
c) supporting coat health in an animal;
d) supporting skin health in an animal;
e) supporting a healthy response to seasonal skin allergy;
f) supporting the immune system;
g) assisting the immune system;
h) enhancing the immune response;
i) providing immune enhancing effects;
j) providing nutritional support for a healthy immune system;
k) supporting a healthy immune system;
l) supporting immune system function;
m) supporting and promote long-term health;
n) promoting the body's innate resistance to pathogens;
o) reducing oxidative stress;
p) providing antioxidant support;
q) helping maintain a normal inflammatory response;
r) maintaining a normal inflammatory cytokine cascade; or
s) a combination thereof.

38. (canceled)

39. A composition comprising CGCC and oclacitinib, or salt thereof, containing composition, wherein oclacitinib is present in free base or salt form.

40. The composition of claim 39, further comprising one or more excipients, and wherein the cardiac glycoside of the CGCC is oleandrin, and the oleandrin is present in an amount of about 0.05 μg to about 100 μg, and the oclacitinib is present in an amount of about 0.5 mg to about 50 mg.
